SEQUENCE LISTING (3 sequences)

SEQ ID NO: 1 - DNA, 294 bp, synthetic construct
  misc_feature 1..294: note = Synthetic: 39K promoter
  source 1..294: mol_type = other DNA
ttcggactgcttgactcgcagcgaaatacaagcgctgttcagggaagccatcaacacgctcaagcacacgatgaacacagaaaacgtctgcgcgcacatgttggacatcgtgtcgtttgagcgtataaaagaatatataagagctaatttaggccatttcacagtaatcaccgacaaatgttcgaagcgtaaggtgtgtcttcatcacaaacgaattgccaggttgttgggcattaaaaaaatatatcatcaagaatacaaacgggttgtttcaaaggtttacaagaagcaaac

SEQ ID NO: 2 - DNA, 325 bp, synthetic construct
  misc_feature 1..325: note = Synthetic: p6.9 promoter
  source 1..325: mol_type = other DNA
aaattccgttttgcgacgatgcagagtttttgaacaggctgctcaaacacatagatccgtacccgctcagtcggatgtattacaatgcagccaataccatgttttacacgactatggaaaactatgccgtgtccaattgcaagttcaacattgaggattacaataacatatttaaggtgatggaaaatattaggaaacacagcaacaaaaattcaaacgaccaagacgagttaaacatatatttgggagttcagtcgtcgaatgcaaagcgtaaaaaatattaataaggtaaaaattacagctacataaattacacaatttaaac

SEQ ID NO: 3 - DNA, 129 bp, synthetic construct
  misc_feature 1..129: note = Synthetic: Polh promoter
  source 1..129: mol_type = other DNA
atcatggagataattaaaatgataaccatctcgcaaataaataagtattttactgttttcgtaacagttttgtaataaaaaaacctataaatattccggattattcataccgtcccaccatcgggcgcg

(19) United States
(12) Patent Application Publication
(10) Pub. No.: US 2025/0084383 A1
(43) Pub. Date: Mar. 13, 2025

(54) LARGE SCALE ADENO-ASSOCIATED VIRUS PRODUCTION SYSTEMS

(71) Applicant: BIOMARIN PHARMACEUTICAL INC., Novato, CA (US)

(72) Inventors: Tomas CINEK, Novato, CA (US); Joseph Charles PELTIER, Novato, CA (US); Juan Jose APONTE-UBILLUS, Novato, CA (US); Daniel BARAJAS, Novato, CA (US); Santosh G. PANDE, Novato, CA (US)

(73) Assignee: BIOMARIN PHARMACEUTICAL INC., Novato, CA (US)

(21) Appl. No.: 18/292,430

(22) PCT Filed: Jul. 22, 2022
(86) PCT No.: PCT/US2022/074046

§ 371 Date: Jan. 26, 2024

Related U.S. Application Data
(60) Provisional application No. 63/226,564, filed on Jul. 28, 2021.
(60) Provisional application No. 63/226,626, filed on Jul. 28, 2021.

(51) Int. Cl. C12N 7/00; C12N 5/07

(52) U.S. Cl. CPC C12N 7/00; C12N 5/0601; C12N 2710/14022; C12N 2710/14044; C12N 2750/14143; C12N 2750/14152

(57) ABSTRACT

The present invention provides processes for producing and characterizing adenoassociated virus particles and baculovirus particles. The present invention is directed to methods of improving adeno associated virus (AAV) production. The present invention addresses the problems associated with the production of rAAV using baculovirus infected Sf9 cells and achieves an improved method for producing rAAV. The present invention developed different methods for producing recombinant baculovirus (rBV) and recombinant adeno-associated virus (rAA V). These methods address issues such as genome instability and also result in improved production of rAA V, as well as produce rAA V with improved properties.

The Sequence Listing concurrently submitted herewith as an XML file named “6439-0113PWO1” created on Jul. 21, 2022, and having a size of 6 KB is herein incorporated by reference pursuant to 37 C.F.R. § 1.52(e)(5).

### FIELD OF THE INVENTION

The present invention is directed to methods and processes for producing adeno-associated virus (AAV) particles.

### BACKGROUND OF THE INVENTION

The present invention is directed to methods of improving adeno associated virus (AAV) production. AAV are non-enveloped viruses with single-stranded DNA genome with at least one inverted terminal repeat (ITR) at the termini. For example, the AAV2 serotype can have a single-stranded DNA genome of approximately 4.7-kilobases (kb), with two 145 nucleotide-long inverted terminal repeats (ITRs) at the termini. The virus does not encode a polymerase and therefore relies on cellular polymerases for genome replication. The ITRs flank the two viral genes—rep (replication) and cap (capsid), encoding non-structural and structural proteins, respectively. The Rep gene, through the use of two promoters and alternative splicing, encodes four regulatory proteins that are dubbed Rep78, Rep68, Rep52 and Rep40. These proteins are involved in AAV genome replication and packaging. The Cap gene, through alternative splicing and initiation of translation, gives rise to three capsid proteins, VP1 (virion protein 1), VP2 and VP3. The molecular weight of VP1, VP2, and VP3 for AAV2 is 87, 72 and 62 kDa, respectively. These capsid proteins assemble into a near-spherical protein shell of 60 subunits.

AAV are unable to replicate on their own and require co-infection with a helper virus, typically adenovirus or herpesvirus. When AAV infects a human cell alone, its gene expression program is auto-repressed and latent infection of the cell occurs. However, when a latently infected cell is co-infected with a helper virus, such as adenovirus or herpes simplex virus, AAV gene expression is activated leading to excision of the provirus DNA from the host cell chromosome, followed by replication and packaging of the viral genome.

Baculoviruses containing AAV genes and a therapeutic gene have been used to infect Sf9 cells to produce AAV capsids containing the therapeutic gene. For example, baculoviruses containing either AAV Rep/Cap genes or the therapeutic gene are used to co-infect Sf9 cells. With this method, the baculovirus plays a dual role, functioning as the ‘helper’ virus required for AAV production, as well as the vehicle for the AAV and therapeutic genetic material.

The baculovirus AAV production method offers a number of advantages. First, Sf9 cells can be cultivated at high densities as free-floating suspensions in large bioreactors with volumes of up to 2000 litres (L), enabling more efficient AAV production than is achieved with adherent cell cultures. In addition, the Sf9 cells can be grown under serum-free conditions, which improves biosafety by eliminating the presence of potentially immunogenic or toxic animal-derived proteins. Further, baculoviruses cannot replicate in human cells.

However, there are also significant limitations to baculovirus/Sf9 production of AAV capsids. For example, the baculovirus genome can become unstable from multiple passages. If the rep gene is lost, production of the rAAV will stop. Pijlman (Pijlman, Gorben P., et al. “Autographa calfornica baculoviruses with large genomic deletions are rapidly generated in infected insect cells” Virology 283.1 (2001): 132-138) discloses deletions in baculovirus genome specifically within two passages. Airenne (Airenne, Kari J., et al. “Improved generation of recombinant baculovirus genomes in Escherichia coli” Nucleic acids research 31.17 (2003): e101-el01) discloses lethal selection strategies for selecting E. coli colonies with recombinant bacmids. Scholz and Suppmann (Scholz, Judith, and Sabine Suppmann. “A new single-step protocol for rapid baculovirus-driven protein production in insect cells” BMC biotechnology 17.1 (2017): 83) discloses bacmid transfection in suspension and isolating P0 baculovirus to reduce production times for recombinant proteins, but does not disclose isolating P0 BV to address genomic instability or for use in AAV production. Negrete (Negrete, Alejandro, et al. “Economized large-scale production of high yield of rAAV for gene therapy applications exploiting baculovirus expression system” The Journal of Gene Medicine: A cross-disciplinary journal for research on the science of gene transfer and its clinical applications 9.11 (2007): 938-948) discloses infecting Sf cells with baculovirus at a MOI of 0.03 to produce AAV. Mena (Mena, Jimmy A., et al. “Improving adeno-associated vector yield in high density insect cell cultures” The Journal of Gene Medicine: A cross-disciplinary journal for research on the science of gene transfer and its clinical applications 12.2 (2010): 157-167) also discloses infecting Sf cells with baculovirus at a MOI of 0.3 to produce AAV. Neither Negrete nor Mena suggest using less than 0.3 BV MOI for AAV production.

### SUMMARY OF THE INVENTION

The present invention addresses the problems associated with the production of rAAV using baculovirus infected Sf9 cells and achieves an improved method for producing rAAV. The inventors have developed different methods for producing recombinant baculovirus (rBV) and recombinant adeno-associated virus (rAAV). These methods address issues such as genome instability and also result in improved production of rAAV, as well as produce rAAV with improved properties (e.g., higher infectivity, decreased encapsidated nucleic acid impurities, etc.).

Embodiments of producing rAAV are disclosed.

In various embodiments, a method of producing rAAV comprises the step of infecting cells with at least one rBV. The at least one rBV has nucleotide sequences for generating rAAV. The method further comprises the step of culturing the infected cells to generate rAAV. In this method, the at least one rBV is isolated from at least one cell culture comprising cells transfected with at least one of the nucleotide sequences.

In various embodiments, a method of producing rAAV comprises the step of infecting cells with at least one rBV. The at least one rBV has nucleotide sequences for generating rAAV. The method further comprises the step of culturing the infected cells to generate rAAV. In this method, the at least one rBV, prior to the infecting step, is isolated from at least one cell culture comprising cells having at least a portion of a baculovirus genome. The cells are also transfected with at least one nucleotide sequence that combines with the at least a portion of a baculovirus genome to form a baculovirus genome capable of generating rBV.

In various embodiments, a method of producing rAAV comprises the steps of infecting at least one cell with passage zero (P0) rBV and culturing the infected at least one cell to generate rAAV. The P0 rBV has nucleotide sequences for generating rAAV.

In various embodiments, a method of producing rAAV comprises the step of infecting cells with rBV at a multiplicity of infection (MOI) of less than 0.01. The rBV has nucleotide sequences for generating rAAV. The method also comprises the step of culturing the infected cells to generate rAAV.

In various embodiments, a method of large-scale rBV based rAAV production using at least one rBV is disclosed. The method comprises the steps of creating banks of recombinant Escherichia coli (E. coli) containing bacmids with AAV Rep genes, AAV Cap genes, and rAAV vector genomes; cryopreserving said E. coli banks; thawing said E. coli banks; isolating bacmids from the thawed E. coli banks; transfecting insect cells with the bacmids from said thawed E. coli banks and culturing the transfected insect cells; isolating rBV from the transfected insect cells; and infecting further insect cells in a bioreactor with the isolated rBV and culturing the infected insect cells to generate rAAV.

Other embodiments related to rBV based production of rAAV are also disclosed.

In various embodiments, a method for increasing production of rAAV and reducing nucleic acid impurities encapsidated within the produced rAAV is disclosed. The method comprises the step of infecting different cell cultures with a rBV having a nucleotide sequence for an rAAV vector genome and one or more second rBV having nucleotide sequences encoding Rep and Cap proteins. Each cell culture is infected with the first rBV and the one or more second rBV at different ratios of the first rBV MOI: the one or more second rBV MOI: The method also comprises the steps of isolating rAAV from the different cell cultures, determining the titers of the isolated rAAV from the different cell cultures, determining concentrations of encapsidated nucleic acid impurities within the isolated rAAV from the different cell cultures, and identifying one or more ratio(s) of the first rBV MOI: the one or more second rBV MOI from both determining steps.

In various embodiments, a method for measuring rBV titer comprises the step of infecting indicator cells with rBV. The indicator cells have a reporter nucleotide sequence operably linked to an early or intermediate baculovirus promoter sequence. In other embodiments, the reporter nucleotide sequence is operably linked to a baculovirus derived enhancer sequence. The method also comprises the steps of measuring expression of the reporter nucleotide sequence and determining rBV titer from the expression of the reporter nucleotide sequence.

In various embodiments, a cell for measuring rBV titer comprises a reporter nucleotide sequence operably linked to an early or intermediate baculovirus promoter sequence. The reporter nucleotide sequence and the early or intermediate baculovirus promoter sequence are stably maintained within the cell. In different embodiments, the reporter nucleotide sequence is operably linked to a baculovirus derived enhancer sequence and the baculovirus derived enhancer sequence is stably maintained within the cell.

In various embodiments, a method for generating an indicator cell for measuring rBV titer is disclosed. The method includes the step of transfecting a vector comprising a reporter nucleotide sequence operably linked to an early or intermediate baculovirus promoter into a cell. In other embodiments, the reporter nucleotide sequence is operably linked to a baculovirus derived enhancer sequence.

### BRIEF DESCRIPTION OF THE DRAWINGS

FIG. 1 is a graphical representation showing the AAV titers (vector genome (vg)/milliliter (mL)) produced from rBVs providing/encoding a vg with a gene of interest (GOI), Rep, and Cap. The rBVs were cultured with naive Sf9 cells at a MOI of 0.1, 0.01, 0.001, 0.0001, and 0.00001.

FIGS. 2, 3, and 4 are maps of plasmids used for developing indicator cell lines.

FIG. 2 has a nucleotide sequence encoding enhanced green fluorescent protein (eGFP) operably linked to a 39k promoter sequence. FIG. 3 has a nucleotide sequence encoding eGFP operably linked to a p6.9 promoter sequence. FIG. 4 has a nucleotide sequence encoding eGFP operably linked to a polyhedrin (Polh) promoter sequence.

FIG. 5 shows flow cytometry data of untransfected Sf9 cells. The dotted line is a gate showing green fluorescence, in which ˜0.1% of the cells exhibit fluorescence.

FIG. 6 shows flow cytometry data of Sf9 cells transfected with the plasmid containing an eGFP nucleotide sequence operably linked to a 39k promoter sequence. The cells have not been transfected with rBV. The dotted line is a gate showing green fluorescence, in which ˜0.1% of the cells exhibit fluorescence.

FIGS. 7, 8, and 9 shows flow cytometry data of Sf9 cells transfected with the plasmid containing an eGFP nucleotide sequence operably linked to a 39k promoter sequence, a p6.9 promoter sequence, or a Polh promoter sequence. These cells were infected with rBV. The dotted line is a gate showing green fluorescence. For FIG. 7, 55.5% of the 39k promoter cells exhibited green fluorescence. For FIG. 8, 11% of the p6.9 promoter cells exhibited green fluorescence. For FIG. 9, 2% of the Polh promoter cells exhibited green fluorescence.

FIGS. 10, 11, and 12 are graphical representations showing eGFP expression at times post rBV infection. At 19 hours post rBV infection as shown in FIG. 10, 55.5% of the 39k promoter cells exhibited green fluorescence, 11% of the p6.9 promoter cells exhibited green fluorescence, and 2% of the Polh promoter cells exhibited green fluorescence. At 40 hours post rBV infection as shown in FIG. 11, 65.4% of the 39k promoter cells exhibited green fluorescence, 19% of the p6.9 promoter cells exhibited green fluorescence, and 11% of the Polh promoter cells exhibited green fluorescence. At 68 hours post rBV infection as shown in FIG. 12, 66.3% of the 39k promoter cells exhibited green fluorescence, 19% of the p6.9 promoter cells exhibited green fluorescence, and 15% of the Polh promoter cells exhibited green fluorescence.

FIG. 13 is a graphical representation showing eGFP expression of the 39k promoter cells at times post rBV infection. The percentage of 39k promoter cells expressing eGFP was 41.1% (15 hours), 39.9% (18 hours), 40.7% (24 hours), 68.0% (43 hours), 66.4% (65 hours), 67.3% (70 hours), and 69.3% (94 hours).

FIGS. 14, 15, and 16 are graphical representations comparing expression cells containing nucleotide sequences encoding eGFP or eGFP codon optimized for expression in insect cells. At 20 hours as show in FIG. 14, use of the codon optimized eGFP sequence for 39k promoter cells increased the percentage of cells expressing eGFP from 56.5% to 57.8% and use of the codon optimized eGFP sequence for PolH promoter cells increased the percentage of cells expressing eGFP from 4.0% to 10.5%. At 25 hours as show in FIG. 15, use of the codon optimized eGFP sequence for 39k promoter cells resulted in essentially no difference in the percentage of cells expressing eGFP (56.6% and 55.9%) and use of the codon optimized eGFP sequence for PolH promoter cells increased the percentage of cells expressing eGFP from 4.8% to 12.0%. At 48 hours as show in FIG. 16, use of the codon optimized eGFP sequence for 39k promoter cells increased the percentage of cells expressing eGFP from 58.5% to 59.3% and use of the codon optimized eGFP sequence for PolH promoter cells increased the percentage of cells expressing eGFP from 10.9% to 17.5%.

FIGS. 17 and 18 highlight the statistical analysis of the effect of rBV MOI on rAAV5 productivity. FIG. 17 is a graphical representation showing the normalized productivity, values are presented relative to the first condition (GOI 0.01/Rep 0.01/Cap 0.01). FIG. 18 is a graphical representation showing the capsid-to-vg ratio obtained from different experimental cell culture conditions infected with rBV.

FIGS. 19 and 20 show the effect of rBV MOI and rBV co-infection on AAV5 productivity. FIG. 19 is a graphical representation showing productivity among four experimental conditions, using two different rBV transgene sets. For each set, values were normalized relative to its GOI 0.03/Rep 0.003/Cap 0.003 condition. FIG. 20 is a graphical representation showing a comparison between the normalized productivity and Cap copy number (VP3/18s). The peak cell density was adjusted based on the percentage of co-infected cells. For VP3/18s ratio (shown in diamonds), the final value was adjusted based on the percentage of dTomato-expressing cells. The correlation coefficient between the adjusted outputs is 0.849.

FIGS. 21 and 22 show the effect of Rep and Cap BV MOI on packaging of baculovirus DNA impurities. FIG. 21 is a graphical representation showing averaged Alpha-Beta:cp in conditions infected with GOI-B-rBV, Rep rBV, and Cap rBV. FIG. 22 is a graphical representation showing averaged Delta-Gamma:cp in conditions infected with GOI-B-rBV, Rep rBV, and Cap BV. Values are normalized to the first condition, GOI 0.03/Rep 0.003/Cap 0.003. Analysis was performed from nuclease-treated, clarified harvest. GOI-B-rBV-only and Rep Cap-rBV-only, and Cap-rBV-only conditions were included as controls.

### DETAILED DESCRIPTION OF THE INVENTION

As required, detailed embodiments of the present disclosure are disclosed herein; however, it is to be understood that the disclosed embodiments are merely exemplary and may be embodied in various and alternative forms.

Except in the examples, or where otherwise expressly indicated, all numerical quantities in this description indicating amounts of material or conditions of reaction and/or use are to be understood as modified by the word “about”. For example, description referring to “about X” includes description of “X.” In one example, the term “about” is understood as within a range of normal tolerance in the art, for example within 2 standard deviations of the mean. In different examples, “about” refers a variability of ±0.0001%, ±0.0005%, ±0.001%, ±0.005%, ±0.01%, ±0.05%, ±0.1%, ±0.5%, ±1%, ±5%, or ±10%. In further examples, “about” can be understood as within ±9%, ±8, ±7%, ±6%, ±5%, ±4%, ±3%, or ±2%.

The first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

Unless indicated otherwise, all technical and scientific terms used herein have the same meaning as commonly understood by one of ordinary skill in the art to which the present disclosure belongs.

It is also to be understood that this disclosure is not limited to the specific embodiments and methods described below, as specific components and/or conditions may, of course, vary. Furthermore, the terminology used herein is used only for describing particular embodiments and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

The terms “or” and “and” can be used interchangeably and can be understood to mean “and/or”.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

The terms “comprising”, “consisting of”, and “consisting essentially of” can be alternatively used. When one of these three terms is used, the presently disclosed and claimed subject matter can include the use of either of the other two terms.

Throughout this application, where publications are referenced, the disclosures of these publications in their entireties are hereby incorporated by reference into this application to more fully describe the state of the art to which this invention pertains.

The term “heterologous” refers to a polynucleotide sequence that is nonnative to AAV or a cell or is native to AAV or a cell but is not located in its native location or position within the viral genome or host cells genome.

“Encodes,” “encoded” and “encoding” refer to the inherent property of specific sequences of nucleotides in a polynucleotide, such as a gene, a cDNA, or an mRNA, to serve as templates for synthesis of other polymers and macromolecules in biological processes. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA.

The term “expression control element” refers to a nucleic acid sequence in a polynucleotide that is capable of regulating the expression of a nucleotide sequence to which it is operably linked thereto. “Operatively linked” refers to a functional relationship between two parts in which the activity of one-part (e.g., the ability to regulate transcription) results in an action on the other part (e.g., transcription of the sequence). An expression control element is “operably linked” to a nucleotide sequence when the element controls or regulates the transcription or the translation of the nucleotide sequence. Examples of an expression control element includes sequences of promoters (e.g., inducible or constitutive), enhancers, transcription terminators, a start codon (e.g., ATG), splicing signals for introns, stop codons, internal ribosome entry sites, homology region elements (e.g., homology region 2 from Autographa californica multicapsid nucleopolyhedrovirus (AcMNPV)), AAV regulatory elements (e.g., Rep binding element), etc.

The term “promoter” or “promoter polynucleotide” is understood to mean a regulatory sequence/element or control sequence/element that is capable of binding/recruiting an RNA polymerase and initiating transcription of sequence downstream or in a 3′ direction from the promoter. A promoter can be, for example, constitutively active (always on) or inducible in which the promoter is active or inactive in the presence of an external stimulus. The promoter is capable of expressing proteins at high concentration. For example, the transcript level of the promoter is about or is at least about 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 10.5-fold, 11-fold, 11.5-fold, 12-fold, 12.5-fold, 13-fold, 13.5-fold, 14-fold, 14.5-fold, 15-fold, 15.5-fold, 16-fold, 16.5-fold, 17-fold, 17.5-fold, 18-fold, 18.5-fold, 19-fold, 19.5-fold, 20-fold, 50-fold, 100-fold, 250-fold, 500-fold, 1000-fold, 2000-fold, 2500-fold, 3000-fold, 3500-fold, 4000-fold, 4500-fold, 5000-fold, 5500-fold, 6000-fold, 6500-fold, 7000-fold, 7500-fold, 8000-fold, 8500-fold, 9000-fold, 9500-fold, or 10000-fold higher than a transcript level of a native promoter for an operon encoding the regulatory protein. In different examples, the transcript level of the constitutive promoter polynucleotide is a range between any two levels listed above. The promoter can also be positioned to other expression control element(s) to control transcript expression. For example, an expression cassette with a promoter, homology region element, and/or AAV regulatory element can be stably incorporated into the genome of an insect cell such that baculovirus infection of an insect cell induces transcript expression from the expression cassette (See US2012/0100606).

### Adeno-Associated Virus

The therapeutically effective rAAV particles include rAAV particles disclosed in U.S. Pat. No. 9,504,762, WO 2019/222136, US 2019/0376081, and WO 2021/097157, the disclosures of which are hereby incorporated by reference.

“AAV” is a standard abbreviation for adeno-associated virus. Adeno-associated virus is a single-stranded DNA parvovirus having a genome encapsidated by a capsid. There are currently thirteen serotypes of AAV that have been characterized. General information and reviews of AAV can be found in, for example, Carter, 1989, Handbook of Parvoviruses, Vol. 1, pp. 169-228; and Berns, 1990, Virology, pp. 1743-1764, Raven Press, (New York). However, it is fully expected that these same principles will be applicable to additional AAV serotypes since it is well known that the various serotypes are quite closely related, both structurally and functionally, even at the genetic level. (See, e.g., Blacklowe, 1988, pp. 165-174 of Parvoviruses and Human Disease, J. R. Pattison, ed.; and Rose, Comprehensive Virology 3:1-61 (1974)). For example, all AAV serotypes apparently exhibit very similar replication properties mediated by homologous rep genes; and all bear three related capsid proteins. The degree of relatedness is further suggested by heteroduplex analysis which reveals extensive cross-hybridization between serotypes along the length of the genome; and the presence of analogous self-annealing segments at the termini that correspond to inverted terminal repeats (ITRs). The similar infectivity patterns also suggest that the replication functions in each serotype are under similar regulatory control.

An “AAV viral particle” as used herein refers to an infectious viral particle composed of at least one AAV capsid protein and an encapsidated AAV genome. “Recombinant AAV” or “rAAV”, “rAAV virion” or “rAAV viral particle” refers to a viral particle composed of at least one capsid or Cap protein and an encapsidated rAAV vector genome as described herein. Thus, production of rAAV particles includes production of an rAAV vector genome. The rAAV viral particle of different embodiments include AAV particles and rAAV particles disclosed in U.S. Pat. No. 9,504,762, WO 2019/222136, US 2019/0376081, and WO 2021/097157, the disclosures of which are hereby incorporated by reference.

“Capsid” refers to the structure in which the rAAV vector genome is packaged. The capsid includes VP1 proteins or VP3 proteins, but more typically, all three of VP1, VP2, and VP3 proteins, as found in native AAV. The sequence of the capsid proteins determines the serotype of the rAAV virions. rAAV virions include those derived from a number of AAV serotypes, including AAV1, AAV2, AAV4, AAV5, AAV6, AAV7, AAV8, AAV9, AAV10, AAV11, AAV12, AAV13, Bba21, Bba26, Bba27, Bba29, Bba30, Bba31, Bba32, Bba33, Bba34, Bba35, Bba36, Bba37, Bba38, Bba41, Bba42, Bba43, Bba44, Bce14, Bce15, Bce16, Bce17, Bce18, Bce20, Bce35, Bce36, Bce39, Bce40, Bce41, Bce42, Bce43, Bce44, Bce45, Bce46, Bey20, Bey22, Bey23, Bma42, Bma43, Bpo1, Bpo2, Bpo3, Bpo4, Bpo6, Bpo8, Bpo13, Bpo18, Bpo20, Bpo23, Bpo24, Bpo27, Bpo28, Bpo29, Bpo33, Bpo35, Bpo36, Bpo37, Brh26, Brh27, Brh28, Brh29, Brh30, Brh31, Brh32, Brh33, Bfm17, Bfm18, Bfm20, Bfm21, Bfm24, Bfm25, Bfm27, Bfm32, Bfm33, Bfm34, Bfm35, AAV-rh10, AAV-rh39, AAV-rh43, AAVanc80L65, or any variants thereof (see, e.g., U.S. Pat. No. 8,318,480 for its disclosure of non-natural mixed serotypes). Exemplary capsids are also provided in International Application No. WO 2018/022608 and WO 2019/222136, which are incorporated herein in its entirety. The capsid proteins can also be variants of natural VP1, VP2 and VP3, including mutated, chimeric or shuffled proteins. The capsid proteins can be those of rh.10 or other subtype within the various clades of AAV; various clades and subtypes are disclosed, for example, in U.S. Pat. No. 7,906,111. In various embodiments, the capsid of the AAV viral particle has an acetylated or unacetylated VP1, VP2, or VP3 protein with an amino acid sequence that is at least about 50%, 55%, 60%, 65%, 70%, 75%, 80%, 85%, 90%, 95%, 96%, 97%, 98%, 99% or 100% identical to a portion of an amino acid sequence from AAV-1 (Genbank Accession No. AAD27757.1), AAV-2 (NCBI Reference Sequence No. YP_680426.1), AAV-3 (NCBI Reference Sequence No. NP_043941.1), AAV-3B (Genbank Accession No. AAB95452.1), AAV-4 (NCBI Reference Sequence No. NP_044927.1), AAV-5 (NCBI Reference Sequence No. YP_068409.1), AAV-6 (Genbank Accession No. AAB95450.1), AAV-7 (NCBI Reference Sequence No. YP_077178.1), AAV-8 (NCBI Reference Sequence No. YP_077179.1), AAV-9 (Genbank Accession No. AAS99264.1), AAV-10 (Genbank Accession No. AAT46337.1), AAV-11 (Genbank Accession No. AAT46339.1), AAV-12 (Genbank Accession No. AB116639.1), AAV-13 (Genbank Accession No. ABZ10812.1), or any amino acid sequence disclosed in WO 2018/022608 and WO 2019/222136. Construction and use of AAV proteins of different serotypes are discussed in Chao et al., Mol. Ther. 2:619-623, 2000; Davidson et al., PNAS 97:3428-3432, 2000; Xiao et al., J. Virol. 72:2224-2232, 1998; Halbert et al., J. Virol. 74:1524-1532, 2000; Halbert et al., J. Virol. 75:6615-6624, 2001; and Auricchio et al., Hum. Molec. Genet. 10:3075-3081, 2001.

As used herein, an “AAV vector genome”, “vector genome”, or “rAAV vector genome” refers to single-stranded nucleic acids. An rAAV viral particle has an rAAV vector genome encapsidated within a capsid. The rAAV vector genome has an AAV 5′ inverted terminal repeat (ITR) sequence and an AAV 3′ ITR flanking a protein-coding sequence (preferably a functional therapeutic protein-encoding sequence; e.g., FVIII, FIX, and PAH) operably linked to transcription regulatory elements that are heterologous to the AAV viral genome, i.e., one or more promoters and/or enhancers and, optionally, a polyadenylation sequence and/or one or more introns inserted in the regulatory elements or between the regulatory elements and the protein-coding sequence or between exons of the protein-coding sequence. rAAV vector genome refers to nucleic acids that are present in the rAAV virus particle and can be either the sense strand or the anti-sense strand of the nucleic acid sequences disclosed herein. The size of such single-stranded nucleic acids is provided in bases. The terms “inverted terminal repeat” and “ITR” as used herein refers to the art-recognized regions found at the 5′ and 3′ termini of the rAAV genome which function in cis as origins of viral DNA replication and as packaging signals for the viral genome. AAV ITRs, together with the Rep proteins, provide for efficient excision and rescue from, and integration of a nucleotide sequence interposed between two flanking ITRs into a host cell genome. Sequences of certain AAV-associated ITRs are disclosed by Yan et al., J. Virol. 79(1):364-379 (2005). ITRs are also found in a “flip” or “flop” configuration in which the sequence between the AA′ inverted repeats (that form the arms of the hairpin) are present in the reverse complement (Wilmott, Patrick, et al. Human gene therapy methods 30.6 (2019): 206-213). Construction and use of AAV vector genomes of different serotypes are discussed in Chao et al., Mol. Ther. 2:619-623, 2000; Davidson et al., PNAS 97:3428-3432, 2000; Xiao et al., J. Virol. 72:2224-2232, 1998; Halbert et al., J. Virol. 74:1524-1532, 2000; Halbert et al., J. Virol. 75:6615-6624, 2001; and Auricchio et al., Hum. Molec. Genet. 10:3075-3081, 2001. Because of wide construct availability and extensive characterization, illustrative AAV vector genomes disclosed below are derived from serotype 2.

A therapeutically effective rAAV particle or therapeutic rAAV is capable of infecting cells such that the infected cells express (e.g. by transcription and/or by translation) an element (e.g. nucleotide sequence, protein, etc.) of interest. To this extent, the therapeutically effective rAAV particles can include AAV particles having capsids or vector genomes (vgs) with different properties. For example, the therapeutically effective AAV particles can have capsids with different posttranslational modifications. In other examples, the therapeutically effective AAV particles can contain vgs with differing sizes/lengths, plus or minus strand sequences, different flip/flop ITR configurations flip/flop, flop/flip, flip/flip, flop/flop, etc.), different number of ITRs (1, 2, 3, etc.), or truncations. For example, annealing/complementation of overlapping truncated plus and minus genomes occurs in AAV infected cells such that a “complete” nucleic acid encoding the large protein is generated, thereby reconstructing a functional, full-length gene. Therapeutically effective AAV particles are also referred to as “heavy” or “full” capsids.

As an example, a “therapeutic rAAV”, which refers to an rAAV virion, rAAV viral particle, rAAV vector particle, or rAAV that comprises a heterologous polynucleotide that encodes a therapeutic protein, can be used to replace or supplement the protein in vivo. The “therapeutic protein” is a polypeptide that has a biological activity that replaces or compensates for the loss or reduction of activity of a corresponding endogenous protein. For example, a functional phenylalanine hydroxylase (PAH) is a therapeutic protein for phenylketonuria (PKU). Thus, for example recombinant AAV PAH virus can be used for a medicament for the treatment of a subject suffering from PKU. The medicament may be administered by intravenous (IV) administration and the administration of the medicament results in expression of PAH protein in the subject at levels sufficient to alter the neurotransmitter metabolite or neurotransmitter levels in the subject. Optionally, the medicament may also comprise a prophylactic and/or therapeutic corticosteroid for the prevention and/or treatment of any hepatotoxicity associated with administration of the rAAV encoding PAH. The medicament comprising a prophylactic or therapeutic corticosteroid treatment may comprise at least 5, 10, 15, 20, 25, 30, 35, 40, 45, 50, 55, 60, or more mg/day of the corticosteroid. The medicament comprising a prophylactic or therapeutic corticosteroid may be administered over a continuous period of at least about 3, 4, 5, 6, 7, 8, 9, 10 weeks, or more. The PKU therapy may optionally also include tyrosine supplements.

The transgene incorporated into the AAV capsid is not limited and may be any heterologous gene of therapeutic interest. The transgene is a nucleic acid sequence, heterologous to the AAV ITR sequences flanking the transgene, which encodes a polypeptide, protein, or other product, of interest. The nucleic acid coding sequence is operatively linked to regulatory components in a manner which permits transgene transcription, translation, and/or expression in a host cell.

The composition of the transgene sequence will depend upon the use to which the resulting virus will be put. For example, one type of transgene sequence includes a reporter sequence, which upon expression produces a detectable signal. Such reporter sequences include, without limitation, DNA sequences encoding b-lactamase, b-galactosidase (LacZ), alkaline phosphatase, thymidine kinase, green fluorescent protein (GFP), chloramphenicol acetyltransferase (CAT), luciferase, membrane bound proteins including, for example, CD2, CD4, CD8, the influenza hemagglutinin protein, and others well known in the art, to which high affinity antibodies directed thereto exist or can be produced by conventional means, and fusion proteins comprising a membrane bound protein appropriately fused to an antigen tag domain from, among others, hemagglutinin or Myc.

These coding sequences, when associated with regulatory elements which drive their expression, provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA) and immunohistochemistry. For example, where the marker sequence is the LacZ gene, the presence of cells infected by rAAV encoding the signal is detected by assays for beta-galactosidase activity. Where the transgene is green fluorescent protein or luciferase, the rAAV encoding the signal may be detected by instruments measuring fluorescence or luminescence.

However, the transgene is typically a non-marker sequence encoding a product which is useful in biology and medicine, such as proteins, peptides, RNA, enzymes, dominant negative mutants, or catalytic RNAs. Desirable RNA molecules include tRNA, dsRNA, ribosomal RNA, catalytic RNAs, siRNA, small hairpin RNA, trans-splicing RNA, and antisense RNAs. One example of a useful RNA sequence is a sequence which inhibits or extinguishes expression of a targeted nucleic acid sequence in the treated subject. Typically, suitable target sequences include oncologic targets and viral diseases. See, for examples of such targets the oncologic targets and viruses identified below in the section relating to immunogens.

The transgene may be used to correct or ameliorate gene deficiencies, which may include deficiencies in which normal genes are expressed at less than normal levels or deficiencies in which the functional gene product is not expressed. A preferred type of transgene sequence encodes a therapeutic protein or polypeptide which is expressed in an infected cell. The vector genome may further include multiple transgenes, e.g., to correct or ameliorate a gene defect caused by a multi-subunit protein. In certain situations, a different transgene may be used to encode each subunit of a protein, or to encode different peptides or proteins. This is desirable when the size of the DNA encoding the protein subunit is large, e.g., for an immunoglobulin, the platelet-derived growth factor, or a dystrophin protein. In order for the cell to produce the multi-subunit protein, a cell is infected with the recombinant virus containing each of the different subunits. Alternatively, different subunits of a protein may be encoded by the same transgene. In this case, a single transgene includes the DNA encoding each of the subunits, with the DNA for each subunit separated by an internal ribozyme entry site (IRES). This is desirable when the size of the DNA encoding each of the subunits is small, e.g., the total size of the DNA encoding the subunits and the IRES is less than five kilobases. As an alternative to an IRES, the coding sequences may be separated by sequences encoding a 2A peptide, which self-cleaves in a post-translational event. See, e.g., Donnelly et al, J. Gen. Virol., 78(Pt 1): 13-21 (January 1997); Furler, et al, Gene Ther., 8(11):864-873 (June 2001); Klump et al, Gene Ther., 8(10):8 11-817 (May 2001). This 2A peptide is significantly smaller than an IRES, making it well suited for use when space is a limiting factor. More often, when the transgene is large, consists of multi-subunits, or two transgenes are co-delivered, rAAV carrying the desired transgene(s) or subunits are co-administered to allow them to concatamerize in vivo to form a single vector genome. In such an embodiment, a first AAV may carry an expression cassette which expresses a single transgene and a second AAV may carry an expression cassette which expresses a different transgene for co-expression in the host cell. However, the selected transgene may encode any biologically active product or other product, e.g., a product desirable for study.

Suitable transgenes may be readily selected by one of skill in the art. The selection of the transgene is not considered to be a limitation of this invention. The transgene may be a heterologous protein, and this heterologous protein may be a therapeutic protein. Exemplary therapeutic proteins include, but are not limited to, blood factors, such as b-globin, hemoglobin, tissue plasminogen activator, and coagulation factors; colony stimulating factors (CSF); interleukins, such as IL-1, IL-2, IL-3, IL-4, IL-5, IL-6, IL-7, IL-8, IL-9, etc.; growth factors, such as keratinocyte growth factor (KGF), stem cell factor (SCF), fibroblast growth factor (FGF, such as basic FGF and acidic FGF), hepatocyte growth factor (HGF), insulin-like growth factors (IGFs), bone morphogenetic protein (BMP), epidermal growth factor (EGF), growth differentiation factor-9 (GDF-9), hepatoma derived growth factor (HDGF), myostatin (GDF-8), nerve growth factor (NGF), neurotrophins, platelet-derived growth factor (PDGF), thrombopoietin (TPO), transforming growth factor alpha (TGF-a.), transforming growth factor beta (TGF-.b.), and the like; soluble receptors, such as soluble TNF-a. receptors, soluble VEGF receptors, soluble interleukin receptors (e.g., soluble IL-1 receptors and soluble type II IL-1 receptors), soluble g/d T cell receptors, ligand-binding fragments of a soluble receptor, and the like; enzymes, such as a-glucosidase, imiglucarase, b-glucocerebrosidase, and alglucerase; enzyme activators, such as tissue plasminogen activator; chemokines, such as 1P-10, monokine induced by interferon-gamma (Mig), Groa/IL-8, RANTES, MIP-1a, MIR-1b., MCP-1, PF-4, and the like; angiogenic agents, such as vascular endothelial growth factors (VEGFs, e.g., VEGF121, VEGF165, VEGF-C, VEGF-2), glioma-derived growth factor, angiogenin, angiogenin-2; and the like; anti-angiogenic agents, such as a soluble VEGF receptor; protein vaccine; neuroactive peptides, such as nerve growth factor (NGF), bradykinin, cholecystokinin, gastin, secretin, oxytocin, gonadotropin-releasing hormone, beta-endorphin, enkephalin, substance P, somatostatin, prolactin, galanin, growth hormone-releasing hormone, bombesin, dynorphin, warfarin, neurotensin, motilin, thyrotropin, neuropeptide Y, luteinizing hormone, calcitonin, insulin, glucagons, vasopressin, angiotensin II, thyrotropin-releasing hormone, vasoactive intestinal peptide, a sleep peptide, and the like; thrombolytic agents; atrial natriuretic peptide; relaxin; glial fibrillary acidic protein; follicle stimulating hormone (FSH); human alpha-1 antitrypsin; leukemia inhibitory factor (LIF); tissue factors, luteinizing hormone; macrophage activating factors; tumor necrosis factor (TNF); neutrophil chemotactic factor (NCF); tissue inhibitors of metalloproteinases; vasoactive intestinal peptide; angiogenin; angiotropin; fibrin; hirudin; IF-1 receptor antagonists; and the like. Some other non-limiting examples of protein of interest include ciliary neurotrophic factor (CNTF); brain-derived neurotrophic factor (BDNF); neurotrophins 3 and 4/5 (NT-3 and 4/5); glial cell derived neurotrophic factor (GDNF); aromatic amino acid decarboxylase (AADC); hemophilia related clotting proteins, such as Factor VIII, Factor IX, Factor X; dystrophin, mini-dystrophin, or microdystrophin; lysosomal acid lipase; phenylalanine hydroxylase (PAH); glycogen storage disease-related enzymes, such as glucose-6-phosphatase, acid maltase, glycogen debranching enzyme, muscle glycogen phosphorylase, liver glycogen phosphorylase, muscle phosphofructokinase, phosphorylase kinase (e.g., PHKA2), glucose transporter (e.g., GFUT2), aldolase A, b-enolase, and glycogen synthase; lysosomal enzymes (e.g., beta-N-acetylhexosaminidase A); and any variants thereof. The AAV vector genome also includes conventional control elements or sequences which are operably linked to the transgene in a manner which permits its transcription, translation and/or expression in a cell transfected with the vector or infected with the virus. As used herein, “operably linked” sequences include both expression control sequences that are contiguous with the gene of interest and expression control sequences that act in trans or at a distance to control the gene of interest. Suitable genes include those genes discussed in Anguela et al. “Entering the Modern Era of Gene Therapy”, Annual Rev. of Med. Vol. 70, pages 272-288 (2019) and Dunbar et al., “Gene comes of age”, Science, Vol. 359, Issue 6372, eaan4672 (2018).

Expression control sequences can be linked to the transgenes. Examples of expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters which are native, constitutive, inducible and/or tissue-specific, are known in the art and may be utilized. Examples of constitutive promoters include, without limitation, the retroviral Rous sarcoma virus (RSV) LTR promoter (optionally with the RSV enhancer), the cytomegalovirus (CMV) promoter (optionally with the CMV enhancer) (see, e.g., Boshart el al, Cell, 41:521-530 (1985)), the SV40 promoter, the dihydrofolate reductase promoter, the b-actin promoter, the phosphoglycerol kinase (PGK) promoter, and the EF1 promoter. Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Many other systems have been described and can be readily selected by one of skill in the art. Examples of inducible promoters regulated by exogenously supplied compounds, include, the zinc-inducible sheep metallothionine (MT) promoter, the dexamethasone (Dex)-inducible mouse mammary tumor virus (MMTV) promoter, the T7 polymerase promoter system [WO 98/10088]; the ecdysone insect promoter [No et al, Proc. Natl. Acad. Sci. USA, 93:3346-3351 (1996)], the tetracycline-repressible system [Gossen et al., Proc. Natl. Acad. Sci. USA, 89:5547-5551 (1992)], the tetracycline-inducible system [Gossen et al., Science, 268:1766-1769 (1995), see also Harvey et al., Curr. Opin. Chem. Biol., 2:512-518 (1998)], the RU486-inducible system [Wang et al., Nat. Biotech., 15:239-243 (1997) and Wang et al., Gene Ther., 4:432-441 (1997)] and the rapamycin-inducible system [Magari et al., J. Clin. Invest., 100:2865-2872 (1997)]. Other types of inducible promoters, which may be useful in this context, are those which are regulated by a specific physiological state, e.g., temperature, acute phase, a particular differentiation state of the cell, or in replicating cells only.

Optionally, the native promoter for the transgene may be used. The native promoter may be preferred when it is desired that expression of the transgene should mimic the native expression. The native promoter may be used when expression of the transgene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

The transgene may also include a gene operably linked to a tissue specific promoter. For instance, if expression in skeletal muscle is desired, a promoter active in muscle should be used. These include the promoters from genes encoding skeletal b-actin, myosin light chain 2A, dystrophin, muscle creatine kinase, as well as synthetic muscle promoters with activities higher than naturally-occurring promoters (see Li et al., Nat. Biotech., 17:241-245 (1999)). Examples of promoters that are tissue-specific are known for liver (albumin, Miyatake et al., J. Virol., 71:5124-32 (1997); hepatitis B virus core promoter, Sandig et al., Gene Ther., 3:1002-9 (1996); alpha-fetoprotein (AFP), Arbuthnot et al., Hum. Gene Ther., 7:1503-14 (1996)), bone osteocalcin (Stein et al., Mol. Biol. Rep., 24:185-96 (1997)); bone sialoprotein (Chen et al., J. Bone Miner. Res., 11:654-64 (1996)), lymphocytes (CD2, Hansal et al., J. Immunol., 161:1063-8 (1998); immunoglobulin heavy chain; T cell receptor chain), neuronal such as neuron-specific enolase (NSE) promoter (Andersen et al., Cell. Mol. Neurobiol., 13:503-15 (1993)), neurofilament light-chain gene (Piccioli et al., Proc. Natl. Acad. Sci. USA, 88:5611-5 (1991)), and the neuron-specific vgf gene (Piccioli et al., Neuron, 15:373-84 (1995)), among others.

The recombinant AAV can be used to produce a protein of interest in vitro, for example, in a cell culture. For example, the AAV can be used in a method for producing a protein of interest in vitro, where the method includes providing a recombinant AAV comprising a nucleotide sequence encoding the heterologous protein; and contacting the recombinant AAV with a cell in a cell culture, whereby the recombinant AAV expresses the protein of interest in the cell. The size of the nucleotide sequence encoding the protein of interest can vary. For example, the nucleotide sequence can be at least about 0.1 kilobases (kb), at least about 0.2 kb, at least about 0.3 kb, at least about 0.4 kb, at least about 0.5 kb, at least about 0.6 kb, at least about 0.7 kb, at least about 0.8 kb, at least about 0.9 kb, at least about 1 kb, at least about 1.1 kb, at least about 1.2 kb, at least about 1.3 kb, at least about 1.4 kb, at least about 1.5 kb, at least about 1.6 kb, at least about 1.7 kb, at least about 1.8 kb, at least about 2.0 kb, at least about 2.2 kb, at least about 2.4 kb, at least about 2.6 kb, at least about 2.8 kb, at least about 3.0 kb, at least about 3.2 kb, at least about 3.4 kb, at least about 3.5 kb in length, at least about 4.0 kb in length, at least about 5.0 kb in length, at least about 6.0 kb in length, at least about 7.0 kb in length, at least about 8.0 kb in length, at least about 9.0 kb in length, or at least about 10.0 kb in length. In some embodiments, the nucleotide is at least about 1.4 kb in length.

The recombinant AAV can also be used to produce a protein of interest in vivo, for example in an animal such as a mammal. Some embodiments provide a method for producing a protein of interest in vivo, where the method includes providing a recombinant AAV comprising a nucleotide sequence encoding the protein of interest; and administering the recombinant AAV to the subject, whereby the recombinant AAV expresses the protein of interest in the subject. The subject can be, in some embodiments, a non-human mammal, for example, a monkey, a dog, a cat, a mouse, or a cow. The size of the nucleotide sequence encoding the protein of interest can vary. For example, the nucleotide sequence can be at least about 0.1 kb, at least about 0.2 kb, at least about 0.3 kb, at least about 0.4 kb, at least about 0.5 kb, at least about 0.6 kb, at least about 0.7 kb, at least about 0.8 kb, at least about 0.9 kb, at least about 1 kb, at least about 1.1 kb, at least about 1.2 kb, at least about 1.3 kb, at least about 1.4 kb, at least about 1.5 kb, at least about 1.6 kb, at least about 1.7 kb, at least about 1.8 kb, at least about 2.0 kb, at least about 2.2 kb, at least about 2.4 kb, at least about 2.6 kb, at least about 2.8 kb, at least about 3.0 kb, at least about 3.2 kb, at least about 3.4 kb, at least about 3.5 kb in length, at least about 4.0 kb in length, at least about 5.0 kb in length, at least about 6.0 kb in length, at least about 7.0 kb in length, at least about 8.0 kb in length, at least about 9.0 kb in length, or at least about 10.0 kb in length. In some embodiments, the nucleotide is at least about 1.4 kb in length.

Of particular interest is the use of recombinant AAV to express one or more therapeutic proteins to treat various diseases or disorders. Non-limiting examples of the diseases include cancer such as carcinoma, sarcoma, leukemia, lymphoma; and autoimmune diseases such as multiple sclerosis. Non-limiting examples of carcinomas include esophageal carcinoma; hepatocellular carcinoma; basal cell carcinoma, squamous cell carcinoma (various tissues); bladder carcinoma, including transitional cell carcinoma; bronchogenic carcinoma; colon carcinoma; colorectal carcinoma; gastric carcinoma; lung carcinoma, including small cell carcinoma and non-small cell carcinoma of the lung; adrenocortical carcinoma; thyroid carcinoma; pancreatic carcinoma; breast carcinoma; ovarian carcinoma; prostate carcinoma; adenocarcinoma; sweat gland carcinoma; sebaceous gland carcinoma; papillary carcinoma; papillary adenocarcinoma; cystadenocarcinoma; medullary carcinoma; renal cell carcinoma; ductal carcinoma in situ or bile duct carcinoma; choriocarcinoma; seminoma; embryonal carcinoma; Wilm's tumor; cervical carcinoma; uterine carcinoma; testicular carcinoma; osteogenic carcinoma; epithelieal carcinoma; and nasopharyngeal carcinoma. Non-limiting examples of sarcomas include fibrosarcoma, myxosarcoma, liposarcoma, chondrosarcoma, chordoma, osteogenic sarcoma, osteosarcoma, angiosarcoma, endothelio sarcoma, lymphangiosarcoma, lymphangioendotheliosarcoma, synovioma, mesothelioma, Ewing's sarcoma, leiomyosarcoma, rhabdomyosarcoma, and other soft tissue sarcomas. Non-limiting examples of solid tumors include glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma. Non-limiting examples of leukemias include chronic myeloproliferative syndromes; acute myelogenous leukemias; chronic lymphocytic leukemias, including B-cell CLL, T-cell CLL prolymphocytic leukemia, and hairy cell leukemia; and acute lymphoblastic leukemias. Examples of lymphomas include, but are not limited to, B-cell lymphomas, such as Burkitt's lymphoma; Hodgkin's lymphoma; and the like.

Other non-liming examples of the diseases that can be treated using rAAV and methods disclosed herein include genetic disorders including sickle cell anemia, cystic fibrosis, lysosomal acid lipase (LAL) deficiency 1, Tay-Sachs disease, Phenylketonuria, Mucopolysaccharidoses, Glycogen storage diseases (GSD, e.g., GSD types I, II, III, IV, V, VI, VII, VIII, IX, X, XI, XII, XIII, and XIV), Galactosemia, muscular dystrophy (e.g., Duchenne muscular dystrophy), and hemophilia such as hemophilia A (classic hemophilia) and hemophilia B (Christmas Disease), Wilson's disease, Fabry Disease, Gaucher Disease hereditary angioedema (HAE), and alpha 1 antitrypsin deficiency. In addition, the rAAV and methods disclosed herein can be used to treat other disorders that can be treated by local expression of a transgene in the liver or by expression of a secreted protein from the liver or a hepatocyte.

The amount of the heterologous protein expressed in the subject (e.g., the serum of the subject) can vary. For example, in some embodiments the protein can be expressed in the serum of the subject in the amount of at least about 9 milligram (mg)/mL, at least about 10 mg/mL, at least about 11 mg/mL, at least about 12 mg/mL, at least about 13 mg/mL, at least about 14 mg/mL, at least about 15 mg/mL, at least about 16 mg/mL, at least about 17 mg/mL, at least about 18 mg/mL, at least about 19 mg/mL, at least about 20 mg/mL, at least about 21 mg/mL, at least about 22 mg/mL, at least about 23 mg/mL, at least about 24 mg/mL, at least about 25 mg/mL, at least about 26 mg/mL, at least about 27 mg/mL, at least about 28 mg/mL, at least about 29 mg/mL, at least about 30 mg/mL, at least about 31 mg/mL, at least about 32 mg/mL, at least about 33 mg/mL, at least about 34 mg/mL, at least about 35 mg/mL, at least about 36 mg/mL, at least about 37 mg/mL, at least about 38 mg/mL, at least about 39 mg/mL, at least about 40 mg/mL, at least about 41 mg/mL, at least about 42 mg/mL, at least about 43 mg/mL, at least about 44 mg/mL, at least about 45 mg/mL, at least about 46 mg/mL, at least about 47 mg/mL, at least about 48 mg/mL, at least about 49 mg/mL, or at least about 50 mg/mL. The protein of interest may be expressed in the serum of the subject in the amount of about 9 picograms (pg)/mL, about 10 μg/mL, about 50 μg/mL, about 100 μg/mL, about 200 μg/mL, about 300 μg/mL, about 400 μg/mL, about 500 μg/mL, about 600 μg/mL, about 700 μg/mL, about 800 μg/mL, about 900 μg/mL, about 1000 μg/mL, about 1500 μg/mL, about 2000 μg/mL, about 2500 μg/mL, or a range between any two of these values. A skilled artisan will understand that the expression level in which a protein of interest is needed for therapeutic efficacy can vary depending on non-limiting factors, such as the particular protein of interest and the subject receiving the treatment, and an effective amount of the protein can be readily determined by a skilled artisan using conventional methods known in the art without undue experimentation.

### Methods of Producing Adeno-Associated Virus

The present disclosure provides materials and methods for producing rAAV virions in cells such as insect cells.

Methods of making AAV viral particles are described in e.g., U.S. Pat. Nos. U.S. Pat. Nos. 6,204,059, 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508, 5,064,764, 6,194,191, 6,566,118, 8,137,948; or International Publication Nos. WO1996039530, WO1998010088, WO1999014354, WO1999015685, WO1999047691, WO2000055342, WO2000075353, WO2001023597, WO2015191508, WO2019217513, WO2018022608, WO2019222136, WO2020232044, WO2019222132; Methods In Molecular Biology, ed. Richard, Humana Press, NJ (1995); O'Reilly et al., Baculovirus Expression Vectors, A Laboratory Manual, Oxford Univ. Press (1994); Samulski et al., J. Vir.63:3822-8 (1989); Kajigaya et al., Proc. Nat'l. Acad. Sci. USA 88: 4646-50 (1991); Ruffing et al., J. Vir.66:6922-30 (1992); Kimbauer et al., Vir., 219:37-44 (1996); Zhao et al., Vir.272:382-93 (2000); the contents of each of which are herein incorporated by reference in their entirety.

Cells such as, e.g., an insect cell, yeast cell, and mammalian cell (e.g., human cell or non-human mammalian cell) are capable of generating rAAV. For example, cells are capable of generating rAAV when provided AAV helper functions, AAV non-helper functions, and a nucleotide sequence that the cells use to generate an AAV vector genome. In various embodiments, the AAV helper functions, AAV non-helper functions, and a nucleotide sequence that the cells use to generate rAAV are provided by a vector that is delivered to cell, for example, via transfection with transfection reagents, via transductions/infections with other recombinant viruses, by incorporating nucleotide sequences into the genomes of the cells, or by other methods.

The term “vector” is understood to refer to any genetic element, such as a plasmid, phage, transposon, cosmid, bacmid, mini-plasmid (e.g., plasmid devoid of bacterial elements), Doggybone DNA (e.g., minimal, closed-linear constructs), chromosome, virus, virion (e.g., baculovirus), etc., which is capable of replication when associated with the proper control elements and which can transfer gene sequences between cells. An “insect cell-compatible vector” or “vector” as used herein refers to a nucleic acid molecule capable of productive transformation or transfection of an insect or insect cell. Exemplary biological vectors include plasmids, linear nucleic acid molecules, and recombinant viruses. Any vector can be employed as long as it is insect cell-compatible. The vector may integrate into the insect cells genome but the presence of the vector in the insect cell need not be permanent and transient episomal vectors are also included. The vectors can be introduced by any means known, for example by chemical treatment of the cells, electroporation, or infection. Baculoviral vectors and methods for their use are described in the above cited references on molecular engineering of insect cells.

The vector from which the cell generates an rAAV vector genome may contain a promoter and a restriction site downstream of the promoter to allow insertion of a polynucleotide encoding one or more proteins of interest, wherein the promoter and the restriction site are located downstream of the 5′ AAV ITR and upstream of the 3′ AAV ITR. The vector may also contain a posttranscriptional regulatory element downstream of the restriction site and upstream of the 3′ AAV ITR. The viral construct may further comprise a polynucleotide inserted at the restriction site and operably linked with the promoter, where the polynucleotide comprises the coding region of a protein of interest.

The term “AAV helper” refer to AAV-derived coding sequences which can be expressed to provide AAV gene products that, in turn, function in trans for productive AAV replication. Thus, AAV helper functions include both of the major AAV open reading frames (ORFs), rep and cap. The Rep expression products have been shown to possess many functions, including, among others: recognition, binding and nicking of the AAV origin of DNA replication; DNA helicase activity; and modulation of transcription from AAV (or other heterologous) promoters. The capsid (Cap) expression products supply necessary packaging functions. AAV helper functions are used herein to complement AAV functions in trans that are missing from AAV vector genomes.

In various embodiments, a vector providing AAV helper functions includes a nucleotide sequence(s) that encode capsid proteins or Rep proteins. The cap genes and/or rep gene from any AAV serotype (including, but not limited to, AAV1 (NCBI Reference Sequence No./Genbank Accession No. NC_002077.1), AAV2 (NCBI Reference Sequence No./Genbank Accession No. NC_001401.2), AAV3 (NCBI Reference Sequence No./Genbank Accession No. NC_001729.1), AAV3B (NCBI Reference Sequence No./Genbank Accession No. AF028705.1), AAV4 (NCBI Reference Sequence No./Genbank Accession No. NC_001829.1), AAV5 (NCBI Reference Sequence No./Genbank Accession No. NC_006152.1), AAV6 (NCBI Reference Sequence No./Genbank Accession No. AF028704.1), AAV7 (NCBI Reference Sequence No./Genbank Accession No. NC_006260.1), AAV8 (NCBI Reference Sequence No./Genbank Accession No. NC_006261.1), AAV9 (NCBI Reference Sequence No./Genbank Accession No. AX753250.1), AAV10 (NCBI Reference Sequence No./Genbank Accession No. AY631965.1), AAV11 (NCBI Reference Sequence No./Genbank Accession No. AY631966.1), AAV12 (NCBI Reference Sequence No./Genbank Accession No. DQ813647.1), AAV13 (NCBI Reference Sequence No./Genbank Accession No. EU285562.1), Bba21, Bba26, Bba27, Bba29, Bba30, Bba31, Bba32, Bba33, Bba34, Bba35, Bba36, Bba37, Bba38, Bba41, Bba42, Bba43, Bba44, Bce14, Bce15, Bce16, Bce17, Bce18, Bce20, Bce35, Bce36, Bce39, Bce40, Bce41, Bce42, Bce43, Bce44, Bce45, Bce46, Bey20, Bey22, Bey23, Bma42, Bma43, Bpo1, Bpo2, Bpo3, Bpo4, Bpo6, Bpo8, Bpo13, Bpo18, Bpo20, Bpo23, Bpo24, Bpo27, Bpo28, Bpo29, Bpo33, Bpo35, Bpo36, Bpo37, Brh26, Brh27, Brh28, Brh29, Brh30, Brh31, Brh32, Brh33, Bfm17, Bfm18, Bfm20, Bfm21, Bfm24, Bfm25, Bfm27, Bfm32, Bfm33, Bfm34, Bfm35, AAV-rh10, AAV-rh39, AAV-rh43, AAVanc80L65, or any variants thereof) can be used herein to produce the recombinant AAV Exemplary capsids are also provided in International Application No. WO 2018/022608 and WO 2019/222136, which are incorporated herein in its entirety. Each NCBI Reference Sequence Number or Genbank Accession Numbers provided above is also incorporated by reference herein. In some embodiments, the AAV cap genes encode a capsid from serotype 1, serotype 2, serotype 3, serotype 3B, serotype 4, serotype 5, serotype 6, serotype 7, serotype 8, serotype 9, serotype 10, serotype 11, serotype 12, serotype 13, or a variant thereof.

For production, cells with AAV helper functions produce recombinant capsid proteins sufficient to form a capsid. This includes at least VP1 and VP3 proteins, but more typically, all three of VP1, VP2, and VP3 proteins, as found in native AAV. The sequence of the capsid proteins determines the serotype of the AAV virions produced by the host cell. Capsids useful in the invention include those derived from a number of AAV serotypes, including 1, 2, 3, 3B, 4, 5, 6, 7, 8, 9, 10, 11, 12, 13 or mixed serotypes (see, e.g., U.S. Pat. No. 8,318,480 for its disclosure of non-natural mixed serotypes). The capsid proteins can also be variants of natural VP1, VP2 and VP3, including mutated, chimeric or shuffled proteins. The capsid proteins can be those of rh.10 or other subtype within the various clades of AAV; various clades and subtypes are disclosed, for example, in U.S. Pat. No. 7,906,111. Because of wide construct availability and extensive characterization, illustrative AAV vectors disclosed below are derived from serotype 2. Construction and use of AAV vectors and AAV proteins of different serotypes are discussed in Chao et al., Mol. Ther. 2:619-623, 2000; Davidson et al., PNAS 97:3428-3432, 2000; Xiao et al., J. Virol. 72:2224-2232, 1998; Halbert et al., J. Virol. 74:1524-1532, 2000; Halbert et al., J. Virol. 75:6615-6624, 2001; and Auricchio et al., Hum. Molec. Genet. 10:3075-3081, 2001.

In various embodiments, nucleotide sequences encoding VP proteins can be operably linked to a suitable expression control sequence. For example, the nucleotide sequences can be operably linked to baculoviral promoters such as the Polh promoter, ΔIE1 promoter, p5 promoter, p10 promoter, the p40 promoter, metallothionein promoter, 39K promoter, p6.9 promoter, and orf46 promoter.

In different examples, the 39K promoter includes a nucleotide sequence that is at least 95%, 96%, 97%, 98%, 99%, 99+%, or 100% identical to SEQ ID NO: 1.

TTCGGACTGCTTGACTCGCAGCGAAATACAAGCGCTGTTCAGGGAAGCC
ATCAACACGCTCAAGCACACGATGAACACAGAAAACGTCTGCGCGCACA
TGTTGGACATCGTGTCGTTTGAGCGTATAAAAGAATATATAAGAGCTAA
TTTAGGCCATTTCACAGTAATCACCGACAAATGTTCGAAGCGTAAGGTG
TGTCTTCATCACAAACGAATTGCCAGGTTGTTGGGCATTAAAAAAATAT
ATCATCAAGAATACAAACGGGTTGTTTCAAAGGTTTACAAGAAGCAAAC

In different examples, the p6.9 promoter includes a nucleotide sequence that is at least 95%, 96%, 97%, 98%, 99%, 99+%, or 100% identical to SEQ ID NO: 2.

AAATTCCGTTTTGCGACGATGCAGAGTTTTTGAACAGGCTGCTCAAACA
CATAGATCCGTACCCGCTCAGTCGGATGTATTACAATGCAGCCAATACC
ATGTTTTACACGACTATGGAAAACTATGCCGTGTCCAATTGCAAGTTCA
ACATTGAGGATTACAATAACATATTTAAGGTGATGGAAAATATTAGGAA
ACACAGCAACAAAAATTCAAACGACCAAGACGAGTTAAACATATATTTG
GGAGTTCAGTCGTCGAATGCAAAGCGTAAAAAATATTAATAAGGTAAAA
ATTACAGCTACATAAATTACACAATTTAAAC

In different examples, the Polh promoter includes a nucleotide sequence that is at least 95%, 96%, 97%, 98%, 99%, 99+%, or 100% identical to SEQ ID NO: 3.

ATCATGGAGATAATTAAAATGATAACCATCTCGCAAATAAATAAGTATT
TTACTGTTTTCGTAACAGTTTTGTAATAAAAAAACCTATAAATATTCCG
GATTATTCATACCGTCCCACCATCGGGCGCG

For production, cells with AAV helper functions produce Rep proteins to promote production of rAAV. It has been found that infectious particles can be produced when at least one large Rep protein (Rep78 or Rep68) and at least one small Rep protein (Rep52 and Rep40) are expressed in cells. In a specific embodiment all four of Rep 78, Rep68, Rep52 and Rep 40 are expressed. Alternately, Rep78 and Rep52, Rep78 and Rep40, Rep 68 and Rep52, or Rep68 and Rep40 are expressed. Examples below demonstrate the use of the Rep78/Rep52 combination. Rep proteins can be derived from AAV-2 or other serotypes. In various embodiments, nucleotide sequences encoding Rep proteins can be operably linked to a suitable expression control sequence. For example, the nucleotide sequences can be operably linked to baculoviral promoters such as the polyhedrin (Polh) promoter, ΔIE1 promoter, p5 promoter, p10 promoter, the p40 promoter, metallothionein promoter, 39K promoter, p6.9 promoter, and orf46 promoter.

Cells with AAV helper functions can also produce assembly-activating proteins (AAP), which help assemble capsids. In various embodiments, nucleotide sequences encoding AAP can be operably linked to a suitable expression control sequence. For example, the nucleotide sequences can be operably linked to baculoviral promoters such as the polyhedrin (Polh) promoter, ΔIE1 promoter, p5 promoter, p10 promoter, the p40 promoter, metallothionein promoter, 39K promoter, p6.9 promoter, and orf46 promoter.

The term “non-AAV helper function” refers to non-AAV derived viral and/or cellular functions upon which AAV is dependent for its replication. Thus, the term captures proteins and RNAs that are required in AAV replication, including those moieties involved in activation of AAV gene transcription, stage specific AAV mRNA splicing, AAV DNA replication, synthesis of Cap expression products and AAV capsid assembly. Viral-based accessory functions can be derived from any of the known helper viruses such as adenovirus, herpesvirus (other than herpes simplex virus type-1) and vaccinia virus.

The term “non-AAV helper function vector” refers generally to a nucleic acid molecule that includes nucleotide sequences providing accessory functions. An accessory function vector can be transfected into a suitable host cell, wherein the vector is then capable of supporting AAV virion production in the host cell. Expressly excluded from the term are infectious viral particles as they exist in nature, such as adenovirus, herpesvirus or vaccinia virus particles. Thus, accessory function vectors can be in the form of a plasmid, phage, transposon or cosmid. In particular, it has been demonstrated that the full-complement of adenovirus genes are not required for accessory helper functions. For example, adenovirus mutants incapable of DNA replication and late gene synthesis have been shown to be permissive for AAV replication. Ito et al., (1970) J. Gen. Virol. 9:243; Ishibashi et al, (1971) Virology 45:317. Similarly, mutants within the E2B and E3 regions have been shown to support AAV replication, indicating that the E2B and E3 regions are probably not involved in providing accessory functions. Carter et al., (1983) Virology 126:505. However, adenoviruses defective in the E1 region, or having a deleted E4 region, are unable to support AAV replication. Thus, ElA and E4 regions are likely required for AAV replication, either directly or indirectly. Laughlin et al., (1982). J. Virol. 41:868; Janik et al., (1981) Proc. Natl. Acad. Sci. USA 78:1925; Carter et al., (1983) Virology 126:505. Other characterized Ad mutants include: E1B (Laughlin et al. (1982), supra; Janik et al. (1981), supra; Ostrove et al., (1980) Virology 104:502); E2A (Handa et al., (1975) J. Gen. Virol. 29:239; Strauss et al., (1976) J. Virol. 17:140; Myers et al., (1980) J. Virol. 35:665; Jay et al., (1981) Proc. Natl. Acad. Sci. USA 78:2927; Myers et al., (1981) J. Biol. Chem. 256:567); E2B (Carter, Adeno-Associated Virus Helper Functions, in I CRC Handbook of Parvoviruses (P. Tijssen ed., 1990)); E3 (Carter et al. (1983), supra); and E4 (Carter et al. (1983), supra; Carter (1995)). Although studies of the accessory functions provided by adenoviruses having mutations in the E1B coding region have produced conflicting results, Samulski et al., (1988) J. Virol. 62:206-210, recently reported that E1B55k is required for AAV virion production, while E1B19k is not. In addition, International Publication WO 97/17458 and Matshushita et al., (1998) Gene Therapy 5:938-945, describe accessory function vectors encoding various Ad genes. Particularly preferred accessory function vectors comprise an adenovirus VA RNA coding region, an adenovirus E4 ORF6 coding region, an adenovirus E2A 72 kD coding region, an adenovirus ElA coding region, and an adenovirus E1B region lacking an intact E1B55k coding region. Such vectors are described in International Publication No. WO 01/83797.

Use of insect cells for expression of heterologous proteins is well documented, as are methods of introducing nucleic acids, such as vectors, e.g., insect-cell compatible vectors, into such cells and methods of maintaining such cells in culture. (See, e.g., METHODS IN MOLECULAR BIOLOGY, ed. Richard, Humana Press, N J (1995); O'Reilly et al., BACULOVIRUS EXPRESSION VECTORS, A LABORATORY MANUAL, Oxford Univ. Press (1994); Samulski et al., J. Vir. (1989) vol. 63, pp. 3822-3828; Kajigaya et al., Proc. Nat'l. Acad. Sci. USA (1991) vol. 88, pp. 4646-4650; Ruffing et al., J. Vir. (1992) vol. 66, pp. 6922-6930; Kirnbauer et al., Vir. (1996) vol. 219, pp. 37-44; Zhao et al., Vir. (2000) vol. 272, pp. 382-393; and U.S. Pat. No. 6,204,059). Examples of insect cell lines that can be used may be derived from Spodoptera frugiperda, such as Sf9, Sf21, Sf900+, drosophila cell lines, mosquito cell lines, e.g., Aedes albopictus derived cell lines, domestic silkworm cell lines, e.g., Bombyxmori cell lines, Trichoplusia ni cell lines such as High Five cells or Lepidoptera cell lines such as Ascalapha odorata cell lines. Exemplary insect cells are cells from the insect species which are susceptible to baculovirus infection, including High Five, Sf9, Sf-RVN, Se301, SeIZD2109, SeUCR1, Sf900+, Sf21, BTI-TN-5B1-4, MG-1, Tn368, HzAm1, BM-N, Ha2302, Hz2E5 and Ao38.

In various embodiments, insect cells having vectors for rAAV production are provided. Recombinant baculovirus (rBV) with nucleotide sequences for rAAV production can be used to deliver these nucleotide sequences to the insect cells for rAAV production. Baculoviruses, such as rBV, are enveloped DNA viruses of arthropods, two members of which are well known expression vectors for producing recombinant proteins in cell cultures. Baculoviruses have circular double-stranded genomes (80-200 kbp) which can be engineered to allow the delivery of large genomic content to specific cells. The viruses used as a vector are generally Autographa californica multicapsid nucleopolyhedrovirus (AcMNPV) or Bombyx mori nucleopolyhedrovirus (Bm-NPV) (Katou, Yasuhiro, et al., Virology 404.2 (2010): 204-214.). Baculoviruses are commonly used for the infection of insect cells for the expression of recombinant proteins. In particular, expression of heterologous genes in insects can be accomplished as described in for instance U.S. Pat. No. 4,745,051; Friesen, P. D., and L. K. Miller., Current topics in microbiology and immunology 131 (1986): 31-49; EP 127839; EP 155476; Vlak, Just M., et al., Journal of General Virology 69.4 (1988): 765-776; Miller, Lois K., Annual Reviews in Microbiology 42.1 (1988): 177-199; Carbonell, Luis F., et al., Gene 73.2 (1988): 409-418; Maeda, Susumu, et al., Nature 315.6020 (1985): 592-594; Lebacq-Verheyden, ANNE-MARIE, et al., Molecular and cellular biology 8.8 (1988): 3129-3135; Smith, Gale E., et al., Proceedings of the National Academy of Sciences 82.24 (1985): 8404-8408; Miyajima, A tsushi, et al., Gene 58.2-3 (1987): 273-281; and Martin, Brian M., et al., DA 7.2 (1988): 99-106. Numerous baculovirus strains and variants and corresponding permissive insect host cells that can be used for protein production are described in Luckow, Verne A., and Max D. Summers., Bio technology 6.1 (1988): 47-55; Miller et al. (1986) Genetic Engineering, Principles and Mehods, Vol. 8 (eds. J. Setlow and A. Hollaender), Plenum Press, N.Y., pp. 277-298, 1986); Maeda, Susumu, et al., Nature 315.6020 (1985): 592-594; and McKenna, Kevin A., Hluazhu Hong, and Robert R. Granados., Journal of Invertebrate Pathology 71.1 (1998): 82-90.

A donor vector and a bacmid or a transfer vector and linearized baculovirus DNA are used for generating recombinant baculoviruses (rBV). Bacmids propagate in bacteria such as Escherichia coli as a large plasmid. When transfected into insect cells, the bacmids generate baculovirus. Traditional baculovirus generation, e.g. as is one in the Invitrogen's Bac-to-Bac system generates recombinant baculovirus by site-specific transposition in E. coli. High molecular weight bacmid DNA is then isolated and transfected into Sf9 or Sf21 cells from which recombinant baculovirus is isolated and amplified.

Insect cells can be separately transfected with bacmids having nucleotide sequences for rAAV vector genome or having nucleotide sequences providing AAV helper functions to generate rBV. These different rBVs are subsequently used to co-infect naive insect cells to generate rAAV.

A significant problem that exists with currently used protocols for AAV production in insect cells is the instability of the baculovirus. This instability leads to the generation of Defective Interfering Particles (DIPs). The instability is caused, in part, because baculovirus genome replication is inherently unstable resulting in large deletions, including the gene of interest, and resulting in low rAAV productivity. Common mitigation strategies for addressing this problem involve performing passage and infection with rBV at very low multiplicity of infection (MOI). (The MOI refers to the average number of virus particles infecting each cell, i.e. the number of viruses added per cell during infection.) A second strategy is to clone the rBVs to select for stability. Other strategies involve modifications of the baculovirus backbones to try to optimize stability by e.g. repositioning selection markers near critical genes and/or moving/deleting repeating hr regions.

As noted above, one strategy for addressing baculovirus instability involves using a low MOI: While using a low MOI does somewhat improve baculovirus instability on a small scale, the improvement is not sufficient for commercial production.

For example, it was discovered that the baculovirus (BV) genome is unstable and deletions occur in the rep and cap genes or the therapeutic gene after multiple passages. These deletions impair rAAV productivity. To address the deletions and impaired productivity, different processes were developed. The first process is to bank stable E. coli clones with bacmids and propagate the bacmids in the clones. The second process is to conduct transfections of Sf cells in suspension to generate BV and to isolate passage 0 (P0) BV for rAAV production. The third process is to infect Sf cells with P0 BVs at an ultra-low multiplicity of infection (MOI) (e.g., less than 0.01) to produce rAAV.

In various embodiments, a method of producing rAAV comprises the step of infecting cells with at least one recombinant baculovirus (rBV). The at least one rBV has nucleotide sequences for generating rAAV. The method further comprises the step of culturing the infected cells to generate rAAV. In this method, the at least one rBV is isolated from at least one cell culture comprising cells transfected with at least one of the nucleotide sequences. For example, the at least one of the nucleotide sequences are in a bacmid.

In various embodiments, the rAAV is produced using any insect cell type which allows for production of AAV or biologic products and which can be maintained in culture and which is susceptible to baculovirus infection, including, but not limited to, High Five, Sf9, Sf-RVN, Se301, SeIZD2109, SeUCR1, Sf900+, Sf21, BTI-TN-5B1-4, MG-1, Tn368, HzAm1, BM-N, Ha2302, Hz2E5 and Ao38.

In various embodiments, a method of producing rAAV comprises the step of infecting cells with at least one recombinant baculovirus (rBV). The at least one rBV has nucleotide sequences for generating rAAV. The method further comprises the step of culturing the infected cells to generate rAAV. In this method, the at least one rBV, prior to the infecting step, is isolated from at least one cell culture comprising cells having at least a portion of a baculovirus genome. The cells are also transfected with at least one nucleotide sequence that combines with the at least a portion of a baculovirus genome to form a baculovirus genome capable of generating rBV. For example, a linearized baculovirus genome can be recombined with a nucleotide molecule such that a baculovirus genome containing the nucleotide molecule is generated such that cells having the newly formed baculovirus genome are capable of generating rBV.

In various embodiments, a method of producing rAAV comprises the steps of infecting cells with passage zero (P0) rBV and culturing the infected cells to generate rAAV. The P0 rBV has nucleotide sequences for generating rAAV. In other embodiments, rBV used to infect cells for generating rAAV is at a passage less than 1.

In various embodiments, a method of large-scale rBV based rAAV production using at least one rBV is disclosed. The method comprises the steps of creating banks of recombinant E. coli containing bacmids with AAV rep genes, AAV cap genes, and rAAV vector genomes; cryopreserving said E. coli banks; thawing said E. coli banks; isolating bacmids from the thawed E. coli banks; transfecting insect cells with the bacmids from said thawed E. coli banks and culturing the transfected insect cells; isolating rBV from the transfected insect cells; and infecting further insect cells in a bioreactor with the isolated rBV and culturing the infected insect cells to generate rAAV.

The term “passage” as it relates to rBV is the process of propagating rBV concentrations by infecting naive insect cells such as Sf9 cells in culture to generate more rBV. The number associated with the term “passage” refers to the sequential number of times that a bacmid or rBV from a previous passage has been used to generate more rBV. For example, transfecting bacmid to at least a portion of naive Sf9 cells in culture and culturing these cells will produce passage 0 or P0 rBV that is isolated. When the P0 rBV is used to infect at least a portion of naive Sf9 cells in culture, these cells will generate P1 rBV.

In various embodiments, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least +99%, or 100% of P0 rBV generated by methods of various embodiments have nucleotide sequences for generating rAAV.

In various embodiments, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of P0 rBV generated by methods of various embodiments have nucleotide sequences for generating rAAV. In other embodiments, the percentage of P0 rBV generated by methods of various embodiments having nucleotide sequences for generating rAAV is a range between any two percentages provided above.

In various embodiments, the E. coli banks or clones are propagated to a predetermined cell density to extract a predetermined concentration of bacmids to transfect cells in a culture volume of at least 5 milliliter (mL), at least 10 mL, at least 50 mL, at least 100 mL, at least 500 mL, at least 1 liter (L), at least 10 L, at least 50 L, at least 100 L, at least 250 L, at least 500 L, at least 1000 L, at least 1500 L, at least 2000 L, or at least 2500 L.

In various embodiments, the cells after transfection are cultured for about 12 hours, about 24 hours, about 36 hours, about 48 hours, about 72 hours, about 96 hours, about 120 hours, about 144 hours, about 168 hours, about 192 hours, about 216 hours, about 240 hours, or a time between any of these two time points after transfection.

In various embodiments, a method of producing rAAV comprises the step of infecting cells with rBV at a multiplicity of infection (MOI) of less than 0.01. The rBV has nucleotide sequences for generating rAAV. The method also comprises the step of culturing the infected cells to generate rAAV. The nucleotide sequences for generating rAAV of various embodiments include nucleotide sequences providing a rAAV vector genome and encoding Rep and capsid proteins.

In various embodiments, a method of large-scale rBV based rAAV production using at least one rBV is disclosed. The method comprises the steps of separately transfecting insect cells in suspension at a volume with bacmids containing AAV rep genes encoding Rep proteins, AAV cap genes encoding Cap proteins, and nucleotide sequences providing rAAV vector genomes; culturing the transfected insect cell and isolating rBV; infecting further insect cells in a bioreactor with rBV; and culturing the infected insect cells to generate rAAV. The volume of various embodiments is, for example, at least 0.0001 milliliter (mL), at least 0.0005 mL, at least 0.001 mL at least 0.005 mL, at least 0.01 mL at least 0.05 mL, at least 0.1 mL, at least 0.5 mL, at least 1 mL, at least 5 mL, at least 10 mL, at least 20 mL, at least 30 mL, at least 40 mL, at least 50 mL, at least 60 mL, at least 70 mL, at least 80 mL, at least 90 mL, at least 100 mL, at least 200 mL, at least 300 mL, at least 400 mL, or at least 500 mL.

The term “multiplicity of infection” and “MOI” is a ratio of the total number of viral particles added per cell during infection. For example, if 1×10⁶ rBV particles are added to a culture containing 1×10⁶ cells, then the MOI is 1.

In various embodiments, the MOI is less than 0.01, 0.0099, 0.0098, 0.0097, 0.0096, 0.0095, 0.0094, 0.0093, 0.0092, 0.0091, 0.009, 0.0089, 0.0088, 0.0087, 0.0086, 0.0085, 0.0084, 0.0083, 0.0082, 0.0081, 0.008, 0.0079, 0.0078, 0.0077, 0.0076, 0.0075, 0.0074, 0.0073, 0.0072, 0.0071, 0.007, 0.0069, 0.0068, 0.0067, 0.0066, 0.0065, 0.0064, 0.0063, 0.0062, 0.0061, 0.006, 0.0059, 0.0058, 0.0057, 0.0056, 0.0055, 0.0054, 0.0053, 0.0052, 0.0051, 0.005, 0.0049, 0.0048, 0.0047, 0.0046, 0.0045, 0.0044, 0.0043, 0.0042, 0.0041, 0.004, 0.0039, 0.0038, 0.0037, 0.0036, 0.0035, 0.0034, 0.0033, 0.0032, 0.0031, 0.003, 0.0029, 0.0028, 0.0027, 0.0026, 0.0025, 0.0024, 0.0023, 0.0022, 0.0021, 0.002, 0.0019, 0.0018, 0.0017, 0.0016, 0.0015, 0.0014, 0.0013, 0.0012, 0.0011, 0.001, 0.00099, 0.00098, 0.00097, 0.00096, 0.00095, 0.00094, 0.00093, 0.00092, 0.00091, 0.0009, 0.00089, 0.00088, 0.00087, 0.00086, 0.00085, 0.00084, 0.00083, 0.00082, 0.00081, 0.0008, 0.00079, 0.00078, 0.00077, 0.00076, 0.00075, 0.00074, 0.00073, 0.00072, 0.00071, 0.0007, 0.00069, 0.00068, 0.00067, 0.00066, 0.00065, 0.00064, 0.00063, 0.00062, 0.00061, 0.0006, 0.00059, 0.00058, 0.00057, 0.00056, 0.00055, 0.00054, 0.00053, 0.00052, 0.00051, 0.0005, 0.00049, 0.00048, 0.00047, 0.00046, 0.00045, 0.00044, 0.00043, 0.00042, 0.00041, 0.0004, 0.00039, 0.00038, 0.00037, 0.00036, 0.00035, 0.00034, 0.00033, 0.00032, 0.00031, 0.0003, 0.00029, 0.00028, 0.00027, 0.00026, 0.00025, 0.00024, 0.00023, 0.00022, 0.00021, 0.0002, 0.00019, 0.00018, 0.00017, 0.00016, 0.00015, 0.00014, 0.00013, 0.00012, 0.00011, 0.0001, 0.000099, 0.000098, 0.000097, 0.000096, 0.000095, 0.000094, 0.000093, 0.000092, 0.000091, 0.00009, 0.000089, 0.000088, 0.000087, 0.000086, 0.000085, 0.000084, 0.000083, 0.000082, 0.000081, 0.00008, 0.000079, 0.000078, 0.000077, 0.000076, 0.000075, 0.000074, 0.000073, 0.000072, 0.000071, 0.00007, 0.000069, 0.000068, 0.000067, 0.000066, 0.000065, 0.000064, 0.000063, 0.000062, 0.000061, 0.00006, 0.000059, 0.000058, 0.000057, 0.000056, 0.000055, 0.000054, 0.000053, 0.000052, 0.000051, 0.00005, 0.000049, 0.000048, 0.000047, 0.000046, 0.000045, 0.000044, 0.000043, 0.000042, 0.000041, 0.00004, 0.000039, 0.000038, 0.000037, 0.000036, 0.000035, 0.000034, 0.000033, 0.000032, 0.000031, 0.00003, 0.000029, 0.000028, 0.000027, 0.000026, 0.000025, 0.000024, 0.000023, 0.000022, 0.000021, 0.00002, 0.000019, 0.000018, 0.000017, 0.000016, 0.000015, 0.000014, 0.000013, 0.000012, 0.000011, 0.00001, 0.0000099, 0.0000098, 0.0000097, 0.0000096, 0.0000095, 0.0000094, 0.0000093, 0.0000092, 0.0000091, 0.000009, 0.0000089, 0.0000088, 0.0000087, 0.0000086, 0.0000085, 0.0000084, 0.0000083, 0.0000082, 0.0000081, 0.000008, 0.0000079, 0.0000078, 0.0000077, 0.0000076, 0.0000075, 0.0000074, 0.0000073, 0.0000072, 0.0000071, 0.000007, 0.0000069, 0.0000068, 0.0000067, 0.0000066, 0.0000065, 0.0000064, 0.0000063, 0.0000062, 0.0000061, 0.000006, 0.0000059, 0.0000058, 0.0000057, 0.0000056, 0.0000055, 0.0000054, 0.0000053, 0.0000052, 0.0000051, 0.000005, 0.0000049, 0.0000048, 0.0000047, 0.0000046, 0.0000045, 0.0000044, 0.0000043, 0.0000042, 0.0000041, 0.000004, 0.0000039, 0.0000038, 0.0000037, 0.0000036, 0.0000035, 0.0000034, 0.0000033, 0.0000032, 0.0000031, 0.000003, 0.0000029, 0.0000028, 0.0000027, 0.0000026, 0.0000025, 0.0000024, 0.0000023, 0.0000022, 0.0000021, 0.000002, 0.0000019, 0.0000018, 0.0000017, 0.0000016, 0.0000015, 0.0000014, 0.0000013, 0.0000012, 0.0000011, 0.000001, 0.000001, 9e-7, 8e-7, 7e-7, 6e-7, 5e-7, 4e-7, 3e-7, 2e-7, 1e-7, 9e-8, 8e-8, 7e-8, 6e-8, 5e-8, 4e-8, 3e-8, 2e-8, 1e-8, 9e-9, 8e-9, 7e-9, 6e-9, 5e-9, 4e-9, 3e-9, 2e-9, 1e-9, 9e-10, 8e-10, 7e-10, 6e-10, 5e-10, 4e-10, 3e-10, 2e-10, 1e-10. In other embodiments, the MOI is a range between any two MOIs provided. In other embodiments, the number of rBV particles added to the culture is 1 virion, 2 virions, 3 virions, 4 virions, 5 virions, 6 virions, 7 virions, 8 virions, 9 virions, or 10 virions. In other embodiments, the number of rBV particles added to the culture are ranges between 0.01 MOI to 1 virion, 0.01 MOI to 2 virions, 0.01 MOI to 3 virions, 0.01 MOI to 4 virions, 0.01 MOI to 5 virions, 0.01 MOI to 6 virions, 0.01 MOI to 7 virions, 0.01 MOI to 8 virions, 0.01 MOI to 9 virions, 0.01 MOI to 10 virions.

In various embodiments, the use of P0 rBV or rBV MOIs of less than 0.01 results in rAAV titer increased by at least 1%, at least 5%, at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 200%, at least 300%, at least 400%, at least 500%, at least 600%, at least 700%, at least 800%, at least 900%, at least 1000%, at least 5000%, at least 10000%, at least 100000%, at least a 7 log increase, at least a 8 log increase, or at least a 9 log. For example, the increase in rAAV titer is relative to rBV at a passage of ≥1 or an rBV MOI of ≥0.01.

In various embodiments, the rBV used to infect cells for rAAV production include a first rBV having a nucleotide sequence for an rAAV vector genome and one or more second rBV having nucleotide sequences encoding Rep and Cap proteins. In various embodiments, the cells are infected at a ratio of the first rBV MOI: the one or more second rBV MOI ranging from 0.01 to 10.0, 0.05 to 7.5, 0.1 to 5, 0.5 to 5, 0.7 to 3.0, 0.8 to 3.0, 0.9 to 3.0, or 1.0 to 3.0. In other embodiments, the ratio of the first rBV MOI: the one or more second rBV MOI is 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, or 10. In other embodiments, the ratio is a range between any two ratio listed above.

In various embodiments, the rAAV generated by a method of different embodiments has a concentration of encapsidated baculoviral nucleotide sequences that is less than 1E-9 nanograms per nanogram of encapsidated rAAV vector genome, less than 1E-10 nanograms per nanogram of encapsidated rAAV vector genome, less than 1E-11 nanograms per nanogram of encapsidated rAAV vector genome, less than 1E-12 nanograms per nanogram of encapsidated rAAV vector genome, less than 1E-13 nanograms per nanogram of encapsidated rAAV vector genome, less than 1E-14 nanograms per nanogram of encapsidated rAAV vector genome, or less than 1E-15 nanograms per nanogram of encapsidated rAAV vector genome. In other embodiments, rAAV generated by a method of different embodiments has a concentration of encapsidated baculoviral nucleotide sequences that is at least at an acceptable level for regulatory approval by a regulatory agency (e.g., United States Food and Drug Administration (FDA), European Medicines Agency (EMA), etc.)

In various embodiments, the rAAV generated by a method of different embodiments has a concentration of encapsidated baculoviral nucleotide sequences that encodes at least a portion of a baculoviral DNA polymerase that is less than 1E-3 copies per copy of encapsidated rAAV vector genome, less than 1E-4 copies per copy of encapsidated rAAV vector genome, less than 1E-5 copies per copy of encapsidated rAAV vector genome, less than 1E-6 copies per copy of encapsidated rAAV vector genome, less than 1E-7 copies, less than 1E-8 copies per copy of encapsidated rAAV vector genome, less than 1E-9 copies per copy of encapsidated rAAV vector genome, or less than 1E-10 copies per copy of encapsidated rAAV vector genome. In other embodiments, the rAAV generated by a method of different embodiments has a concentration of encapsidated baculoviral nucleotide sequences that encodes at least a portion of a baculoviral DNA polymerase that is at least at an acceptable level for regulatory approval by a regulatory agency (e.g., FDA, EMA, etc.).

In various embodiments, the rAAV generated by a method of different embodiments has a concentration of encapsidated cellular 18S ribosomal RNA (rRNA) gene nucleotide sequences that is less than 1E-3 copies per copy of encapsidated rAAV vector genome, less than 5E-3 copies per copy of encapsidated rAAV vector genome, less than 1E-4 copies per copy of encapsidated rAAV vector genome, less than 5E-4 copies per copy of encapsidated rAAV vector genome, less than 1E-5 copies per copy of encapsidated rAAV vector genome, less than 2E-5 copies per copy of encapsidated AAV vector genome, less than 3E-5 copies per copy of encapsidated rAAV vector genome, less than 4E-5 copies per copy of encapsidated rAAV vector genome, less than 5E-5 copies per copy of encapsidated rAAV vector genome, less than 6E-5 copies per copy of encapsidated rAAV vector genome, less than 7E-5 copies per copy of encapsidated rAAV vector genome, less than 8E-5 copies per copy of encapsidated rAAV vector genome, less than 9E-5 per copy of encapsidated rAAV vector genome, less than 1E-6 copies per copy of encapsidated rAAV vector genome, less than 5E-6 copies per copy of encapsidated rAAV vector genome, less than 1E-7 copies per copy of encapsidated rAAV vector genome, less than 5E-7 copies per copy of encapsidated rAAV vector genome, less than 1E-8 copies per copy of encapsidated rAAV vector genome, less than 5E-8 copies per copy of encapsidated rAAV vector genome. In other embodiments, the rAAV generated by a method of different embodiments has a concentration of encapsidated cellular 18S rRNA gene nucleotide sequences that is at least at an acceptable level for regulatory approval by a regulatory agency (e.g., FDA, EMA, etc.).

In various embodiments, the cells infected with the rBVs are cultured for a pre-determined time period before the rAAV is collected. For example, the rAAV particles can be collected at about 12 hours, about 24 hours, about 36 hours, about 48 hours, about 72 hours, about 96 hours, about 120 hours, about 144 hours, about 168 hours, about 192 hours, about 216 hours, about 240 hours, or a time between any of these two time points after the infection.

In various embodiments, the culturing step of various embodiments (e.g., Sf9 cells or E. coli) occurs in a volume of at least 5 mL, at least 10 mL, at least 20 mL, at least 50 mL, at least 100 mL, at least 500 mL, at least 1 liter (L), at least 10 L, at least 50 L, at least 100 L, at least 250 L, at least 500 L, at least 1000 L, at least 1500 L, at least 2000 L, or at least 2500 L.

In examples, the culturing step (e.g., Sf9 cells or E. coli) can occur in a spin tube(s) or a shake flask(s). In various embodiments, the culturing step of any aspect or embodiment occurs in a volume of 0.0001 mL, 0.0005 mL, 0.001 mL 0.005 mL, 0.01 mL 0.05 mL, 0.1 mL, 0.5 mL, 1 mL, 5 mL, 10 mL, 20 mL, 30 mL, 40 mL, 50 mL, 60 mL, 70 mL, 80 mL, 90 mL, 100 mL, 200 mL, 300 mL, 400 mL, 500 mL, 600 mL, 700 mL, 800 mL, 900 mL, 1 L, 2 L, 3 L, 4 L, or 5 L. In other embodiments, the volume of the culturing step is a range between any two volumes provided above.

In other examples, the culturing step (e.g., Sf9 cells or E. coli) can occur in a bioreactor or bioreactors. In various embodiments, the culturing step of any aspect or embodiment occurs in a volume of 1 L, 2 L, 3 L, 4 L, 5 L, 6L, 7L, 8 L, 9 L, 10 L, 11 L, 12 L, 13 L, 14 L, 15 L, 16 L, 17 L, 18 L, 19 L, 20 L, 21 L, 22 L, 23 L, 24 L, 25 L, 26 L, 27 L, 28 L, 29 L, 30 L, 31 L, 32 L, 33 L, 34 L, 35 L, 36 L, 37 L, 38 L, 39 L, 40 L, 41 L, 42 L, 43 L, 44 L, 45 L, 46 L, 47 L, 48 L, 49 L, 50 L, 51 L, 52 L, 53 L, 54 L, 55 L, 56 L, 57 L, 58 L, 59 L, 60 L, 61 L, 62 L, 63 L, 64 L, 65 L, 66 L, 67 L, 68 L, 69 L, 70 L, 71 L, 72 L, 73 L, 74 L, 75 L, 76 L, 77 L, 78 L, 79 L, 80 L, 81 L, 82 L, 83 L, 84 L, 85 L, 86 L, 87 L, 88 L, 89 L, 90 L, 91 L, 92 L, 93 L, 94 L, 95 L, 96 L, 97 L, 98 L, 99 L, 100 L, 110L, 120L, 130L, 140L, 150L, 160L, 170 L, 180 L, 190 L, 200 L, 210 L, 220 L, 230 L, 240 L, 250 L, 260 L, 270 L, 280 L, 290 L, 300 L, 310 L, 320 L, 330 L, 340 L, 350 L, 360 L, 370 L, 380 L, 390 L, 400 L, 410 L, 420 L, 430 L, 440 L, 450 L, 460 L, 470 L, 480 L, 490 L, 500 L, 510 L, 520 L, 530 L, 540 L, 550 L, 560 L, 570 L, 580 L, 590 L, 600 L, 610 L, 620 L, 630 L, 640 L, 650 L, 660 L, 670 L, 680 L, 690 L, 700 L, 710 L, 720 L, 730 L, 740 L, 750 L, 760 L, 770 L, 780 L, 790 L, 800 L, 810 L, 820 L, 830 L, 840 L, 850 L, 860 L, 870 L, 880 L, 890 L, 900 L, 910 L, 920 L, 930 L, 940 L, 950 L, 960 L, 970 L, 980 L, 990 L, 1000 L, 1010 L, 1020 L, 1030 L, 1040 L, 1050 L, 1060 L, 1070 L, 1080 L, 1090 L, 1100 L, 1110 L, 1120 L, 1130 L, 1140 L, 1150 L, 1160 L, 1170 L, 1180 L, 1190 L, 1200 L, 1210 L, 1220 L, 1230 L, 1240 L, 1250 L, 1260 L, 1270 L, 1280 L, 1290 L, 1300 L, 1310 L, 1320 L, 1330 L, 1340 L, 1350 L, 1360 L, 1370 L, 1380 L, 1390 L, 1400 L, 1410 L, 1420 L, 1430 L, 1440 L, 1450 L, 1460 L, 1470 L, 1480 L, 1490 L, 1500 L, 1510 L, 1520 L, 1530 L, 1540 L, 1550 L, 1560 L, 1570 L, 1580 L, 1590 L, 1600 L, 1610 L, 1620 L, 1630 L, 1640 L, 1650 L, 1660 L, 1670 L, 1680 L, 1690 L, 1700 L, 1710 L, 1720 L, 1730 L, 1740 L, 1750 L, 1760 L, 1770 L, 1780 L, 1790 L, 1800 L, 1810 L, 1820 L, 1830 L, 1840 L, 1850 L, 1860 L, 1870 L, 1880 L, 1890 L, 1900 L, 1910 L, 1920 L, 1930 L, 1940 L, 1950 L, 1960 L, 1970 L, 1980 L, 1990 L, 2000 L, 2010 L, 2020 L, 2030 L, 2040 L, 2050 L, 2060 L, 2070 L, 2080 L, 2090 L, 2100 L, 2110 L, 2120 L, 2130 L, 2140 L, 2150 L, 2160 L, 2170 L, 2180 L, 2190 L, 2200 L, 2210 L, 2220 L, 2230 L, 2240 L, 2250 L, 2260 L, 2270 L, 2280 L, 2290 L, 2300 L, 2310 L, 2320 L, 2330 L, 2340 L, 2350 L, 2360 L, 2370 L, 2380 L, 2390 L, 2400 L, 2410 L, 2420 L, 2430 L, 2440 L, 2450 L, 2460 L, 2470 L, 2480 L, 2490 L, 2500 L, 2510 L, 2520 L, 2530 L, 2540 L, 2550 L, 2560 L, 2570 L, 2580 L, 2590 L, 2600 L, 2610 L, 2620 L, 2630 L, 2640 L, 2650 L, 2660 L, 2670 L, 2680 L, 2690 L, 2700 L, 2710 L, 2720 L, 2730 L, 2740 L, 2750 L, 2760 L, 2770 L, 2780 L, 2790 L, 2800 L, 2810 L, 2820 L, 2830 L, 2840 L, 2850 L, 2860 L, 2870 L, 2880 L, 2890 L, 2900 L, 2910 L, 2920 L, 2930 L, 2940 L, 2950 L, 2960 L, 2970 L, 2980 L, 2990 L, or 3000 L. In other embodiments, the volume of the culturing step is a range between any two volumes provided above.

In various embodiments, the titer of the rBV is determined using a foci/viral plaque assay. This assay first includes the step of infecting cells with serial dilutions of a solution containing rBV. After infection occurs for a predetermined time, the rBV is removed from the cultures and the cells are incubated for a pre-determined time. After the pre-determined time has elapsed, a plaquing media (e.g., containing agarose) is added to the cultures and allowed to harden. The cells are allowed to further incubate for a pre-determined time and the number of plaques are counted after the pre-determined time. The titer is calculated using the following formula

Titer (plaque forming units/mL)=number of plaques×dilution factor×(1/(mL of inoculum/well)

For any process of producing rAAV including the process described above, impurities are also produced or found in compositions with the therapeutically effective rAAV particles. Accordingly, rAAV production impurities can include the therapeutically ineffective rAAV particles, extrinsic high molecular weight DNA, small polynucleotides, proteins, buffer components, etc.

Other embodiments related to rBV based production of rAAV are also disclosed.

In various embodiments, a method for increasing production of rAAV and reducing polynucleotide impurities encapsidated within the produced rAAV is disclosed. The method comprises the step of infecting different cell cultures with a rBV having a nucleotide sequence for an rAAV vector genome and one or more second rBV having nucleotide sequences encoding Rep and Cap proteins. Each cell culture is infected with the first rBV and the one or more second rBV at different ratios of the first rBV multiplicity of infection (MOI): the one or more second rBV MOI: The method also comprises the steps of isolating rAAV from the different cell cultures, determining the titers of the isolated rAAV from the different cell cultures, determining concentrations of encapsidated nucleotide impurities within the isolated rAAV from the different cell cultures, and identifying one or more ratio(s) of the first rBV MOI: the one or more second rBV MOI from both determining steps.

In various embodiments, an indicator cell for measuring rBV titer comprises a reporter nucleotide sequence operably linked to an inducible baculovirus promoter sequence activated by a baculovirus infection. The inducible baculovirus promoter sequence is selected from at least one of an early baculovirus promoter sequence and an intermediate baculovirus promoter sequence. The reporter nucleotide sequence and inducible baculovirus promoter sequence are stably maintained within the indicator cell. The reporter nucleotide sequence of various embodiment and inducible baculovirus promoter sequence of various embodiments are stably maintained within the indicator cell (e.g., episomal expression such as episomal minicircles). In other embodiments, the reporter nucleotide sequence and the inducible baculovirus promoter sequence are stably incorporated into the genome of the indicator cell. In various examples, the reporter nucleotide sequence of different embodiments encodes a reporter protein. Examples of reporter proteins include fluorescent proteins, luminescent proteins, or proteins used in histochemistry (e.g., immunohistochemistry, immunohistochemistry, etc.). Further examples of such proteins include cyan fluorescence protein, green fluorescence protein, yellow fluorescence protein, red fluorescence protein, DsRed, mCherry, luciferase, beta-galactosidase, horseradish peroxidase, alkaline phosphatase, chloramphenicol acetyltransferase, and glucose oxidase. In various examples, inducible baculovirus promoter sequence is selected from at least one of 39K promoter, p6.9 promoter, gp64 promoter, Polh promoter, and p10 promoter. The inducible baculovirus promoter sequence of different embodiments is also be positioned to other expression control element(s) to control transcript expression.

In different examples, the indicator cell comprises a promoter nucleotide sequence that is at least 95%, 96%, 97%, 98%, 99%, 99+%, or 100% identical to SEQ ID NO: 1, 2, or 3.

In various embodiment, the reporter nucleotide sequence of different embodiments is operably linked to a baculovirus derived enhancer sequence and the baculovirus derived enhancer sequence is stably maintained within the indicator cell. The baculovirus derived enhancer sequence of various embodiments is stably maintained within the indicator cell (e.g., episomal expression such as episomal minicircles). In other embodiments, the baculovirus derived enhancer sequence is stably incorporated into the genome of the indicator cell. Examples of baculovirus derived enhancer sequences of various embodiments include homologous region (HR) enhancer sequences such as HR1, HRla, HR2, HR2a, HR3, HR4a, HR4b, HR4c, HR5. For example, an expression cassette with a promoter, homology region element, and/or nucleotide sequence encoding acetyltransferase can be stably incorporated into the genome of an insect cell such that baculovirus infection of an insect cell induces transcript expression from the expression cassette (See US2012/0100606).

In various embodiments, the indicator cell for measuring rBV titer of different embodiments further comprises one or more nucleotide sequence providing or encoding one or more elements for selecting cells with the reporter nucleotide sequence of different embodiments, the inducible baculovirus promoter sequence of different embodiments, or the baculovirus derived enhancer sequence of different embodiments (e.g., positive antibiotic selection, selection markers, etc.). In different examples, one selection element can be for selection in one cell type (e.g., Sf9 cells) and another selection element can be for selection in another cell type (e.g., E. coli). Examples of nucleotide sequences of different embodiments as well as elements for selecting cells of different embodiments are provided below. Examples of eukaryotic selection antibiotics for which resistance genes and elements such as proteins are available include Blasticidin (blasticidin resistance gene (bsr) encoding blasticidin-S deaminase), Geneticin (Neomycin resistance gene (neo) from Tn5 encoding an aminoglycoside 3′-phosphotransferase, APH 311), Hygromycin B (hph gene encoding Hygromycin-B 4-O-kinase), Puromycin (Pac gene encoding a puromycin N-acetyl-transferase), Phleomycin (Sh ble gene), or Zeocin (Sh ble gene). Examples of bacterial selection antibiotics for which resistance genes and elements such as proteins are available include Kanamycin (KanR-Tn5 gene product (aminoglycoside phosphotransferase)), Spectinomycin, Streptomycin, Ampicillin (bla gene encoding beta-lactamase), Carbenicillin, Bleomycin, Erythromycin, Polymyxin B, Tetracycline (Tet^{R}-Tn10 gene encoding Tetracycline repressor protein), and Chloramphenicol.

In various embodiments, a method for generating indicator cell(s) for measuring rBV titer of different embodiments is disclosed. The method includes the step of transfecting a vector comprising a reporter nucleotide sequence operably linked to an inducible baculovirus promoter sequence activated by a baculovirus infection. The reporter nucleotide sequence of different embodiments is operably linked to a baculovirus derived enhancer sequence of different embodiments. In various embodiments, the vector further comprises a resistance nucleotide sequence operably linked to an expression control sequence and the method further comprises the steps of culturing the cell and positively selecting at least one cell, in which the vector is stably maintained. In other embodiments, the method further comprises the steps of culturing the cell, isolating a cell from the culture, and separately culturing the isolated the cell.

In various embodiments, a method for measuring rBV titer comprises the step of infecting cells with rBV. The indicator cells of various embodiments comprise a reporter nucleotide sequence operably linked to an inducible baculovirus promoter sequence activated by a baculovirus infection. The inducible baculovirus promoter sequence selected from at least one of an early baculovirus promoter sequence and an intermediate baculovirus promoter sequence. The reporter nucleotide sequence of different embodiments is operably linked to a baculovirus derived enhancer sequence of different embodiments. The method of various embodiment also comprises the steps of measuring expression of the reporter nucleotide sequence and determining rBV titer from the expression of the reporter nucleotide sequence. For example, the reporter nucleotide sequence is measured using flow cytometry. In other embodiments, the determining step occurs 3 hours or more, 4 hours or more, 5 hours or more, 6 hours or more, 7 hours or more, 8 hours or more, 9 hours or more, 10 hours or more, 11 hours or more, 12 hours or more, 13 hours or more, 14 hours or more, 15 hours or more, 16 hours or more, 17 hours or more, 18 hours or more, 19 hours or more, 20 hours or more after the infecting step.

Generally, vg and capsid (cp) titers may be evaluated in any way that is suitable for measuring the respective vg and capsids. For example, quantitative polymerase chain reaction (qPCR) may be used to measure vg titers and enzyme-linked immunosorbent assay (ELISA) may be used to measure Cp titer. Alternatively, SEC (size-exclusion chromatography)-HPLC may be used to measure the vg and cp titers. In addition, RP (reverse phase)-HPLC assay may be used to evaluate the potential impact of process parameters on VP ratios.

qPCR may be used for vg quantification by quantitative polymerase chain reaction (qPCR) using a standard qPCR system, such as an Applied Biosystems 7500 Fast Real-Time PCR system. Alternatively, digital droplet PCR (ddPCR) may be used for Vg quantification. Primers and probes may be designed to target the DNA of the AAV, allowing its quantification as it accumulates during PCR. Examples of ddPCR are described in Pasi, K. John, et al. “Multiyear Follow-Up of AAV5-hFVIII-SQ Gene Therapy for Hemophilia A.” New England Journal of Medicine 382.1 (2020): 29-40; Regan, John F., et al. “A Rapid Molecular Approach for Chromosomal Phasing.” PloS one 10.3 (2015): e0118270; and Furuta-Hanawa, Birei, Teruhide Yamaguchi, and Eriko Uchida. “Two-Dimensional Droplet Digital PCR as a Tool for Titration and Integrity Evaluation of Recombinant Adeno-Associated Viral Vectors” Human gene therapy methods 30.4 (2019): 127-136. Other systems for vg quantification include SEC, SEC-HPLC, and size exchange chromatography multi-angle light scattering, all of which are described in WO 2021/062164, which is incorporated in its entirety by reference.

The capsid ELISA (cp-ELISA) assay measures intact capsids using, e.g., the AAV5 Capsid ELISA method and may utilize a commercially-available kit (for example, Progen PRAAV5). This kit ELISA employs a monoclonal antibody specific for a conformational epitope on assembled AAV5 or other capsids. Capsids can be captured on a plate-bound monoclonal antibody, followed by subsequent binding of a detection antibody. The assay signal may be generated by addition of conjugated streptavidin peroxidase followed by addition of colorimetric TMB substrate solution, and sulfuric acid to end the reaction. The titers of test samples are interpolated from a four-parameter calibration curve of the target capsid standard. Another system for quantifying capsid titers is SEC-MALS, which are described in WO 2021/062164.

The invention will be further described in the following examples, which do not limit the scope of the invention described in the claims.

### Example 1

Using Passage 0 (P0) rBV at Ultra-Low MOIs for rAAV Production

Bacmid Construction and Production: DNA sequences encoding Rep proteins and capsid proteins and providing an rAAV vector genome having a gene of interest (GOI) were cloned into donor plasmids. The donor plasmids were then used to transform DH10Bac competent E. coli cells to generate bacmids with DNA sequences encoding rep and cap and providing rAAV vector genome flanked by two ITRs with a GOI under a promoter. Bacmids from different E. coli clones were isolated and analyzed via Sanger sequencing techniques to select clones with the correct nucleotide sequences. The E. coli clones with the correct nucleotide sequences were used to generate master bacmid E. coli cell banks and working bacmid E. coli cell banks.

To generate bacmids for P0 rBV production, a vial from either the master bacmid E. coli cell or working bacmid E. coli cell banks was thawed and placed into culture. After culturing the E. coli cells to a predetermined cell density, the cells were concentrated and lysed. The bacmids were isolated from lysed cells using different chromatography and filtration processes.

P0 rBVs Production: For passage 0 (P0) rBV production step, Sf9 cells were placed into culture and expanded to a predetermined cell density. The Sf9 cells were then transfected with the generated bacmids using a transfection reagent. The transfected Sf9 cells were cultured for a predetermined time to generate P0 rBV. The P0 rBVs were analyzed using digital droplet polymerase chain reaction (ddPCR) to determine whether the genomes of P0 rBVs contained deletions of nucleotide sequences for rAAV production. It was noted that subsequent passaging of the rBVs resulted in deletions of the rBV genome containing DNA sequences encoding Rep proteins or capsid proteins or providing an rAAV vector genome having the GOI. At the predetermined time, the rBV were isolated from the cell cultures using centrifugation and stored at <15° C.

rAAV Production: For rAAV production, Sf9 cells were placed into culture and expanded to a predetermined cell density. When the cultured Sf9 cells reached the predetermined cell density, the rBVs containing DNA sequences encoding Rep proteins and capsid proteins and providing an rAAV vector genome having the GOI were added to the cultures. In different rAAV productions, the rBVs were added to the cultures at different MOIs (e.g., the number of rBV particles to the number of cells) selected from a range of 0.1 to 1e-10.

After the Sf9 cells were infected with rBVs, the cells were cultured for a predetermined time to generate rAAV. After the predetermined time passed, the supernatant containing the rAAV was recovered, treated with a nuclease, and filtered using different depth filters. The rAAV were then isolated from the supernatant using affinity chromatography. The use of bacmid E. coli cell banks for generating P0 rBV, propagating bacmids in E. coli and isolating them for transfection, transfecting bacmids in Sf9 cells (≥5 mL) to generate P0 rBV, or infecting Sf9 cells with the rBVs at MOIs of less than 0.01 substantially improved the stability of the rBV and substantially improved production of rAAV in the Sf9 cells as well as the infectivity of the generated rAAV.

Analysis of rBV passage on rAAV production: Using a baculovirus infected insect cell system (BIIC), BIICs containing different passages of rBV were analyzed. Particularly, the BIICs spanning 4 passage levels and providing/encoding a GOI, Rep, and Cap were used to produce rAAV. The BIICs were co-cultured with the naive Sf9 cells at an MOI of 1:500, where each BIIC is understood to be capable of releasing approximately hundreds of rBV. The lowest passage BIICs yields AAV titers of approximately 9.55×10e11 vg/mL. The next passage BIICs yields AAV titers of approximately 1.8×10e11 vg/mL. The next passage BIICs yields AAV titers of approximately 3.8×10e10 vg/mL. The highest passage BIICs yields AAV titers of approximately 5.2×10e9 vg/mL. Accordingly, continuous passaging of rBVs reduce AAV titers, making BIICs insufficient for large scale production of rAAV.

To overcome the limitations associated with BIICs, a novel rAAV production process employing bacmids as a starting material for producing high-titer rBVs was developed. The rBV stocks are generated in-process by transfecting the Sf9 cells in shaken suspension cultures during each production run and yield rBV titers that are sufficient for infecting a 2,000 L production tank at a low MOI: By eliminating the rBV passaging, either in the form of BIIC propagation or rBV stock amplification, rBVs were utilized for infecting the Sf9 production cultures before the rBV inherent instability can significantly affect the resulting rAAV titers. Consequently, rAAV titers were achieved that are comparable to or greater than those attainable with rBVs derived from low passage BIICSs, but without limitations on scalability and supply.

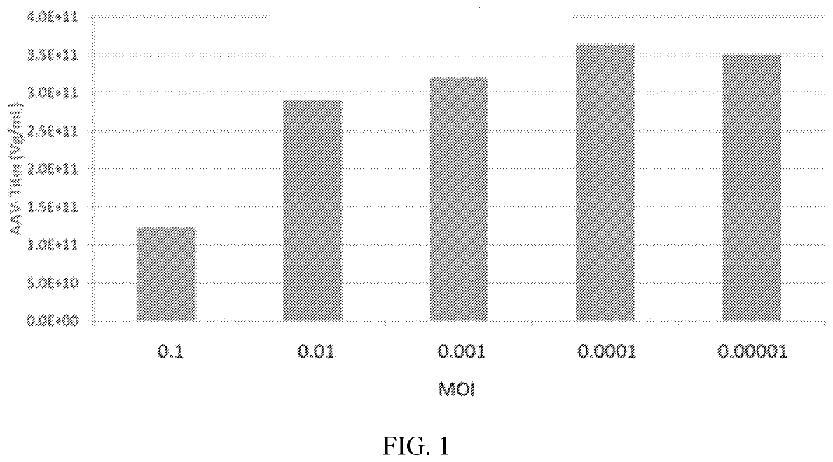

P0 rBVs providing/encoding a GOI, Rep, and Cap were subsequently used to generate rAAV. As shown in FIG. 1, the naive sf9 cells were infected with rBVs at 0.1, 0.01, 0.001, 0.0001 and 0.00001. FIG. 1 shows that infecting Sf9 cell with P0 rBVs at MOIs of <0.01 significantly improved rAAV titers.

### Example 2

### Generation of Indicator Cell Lines

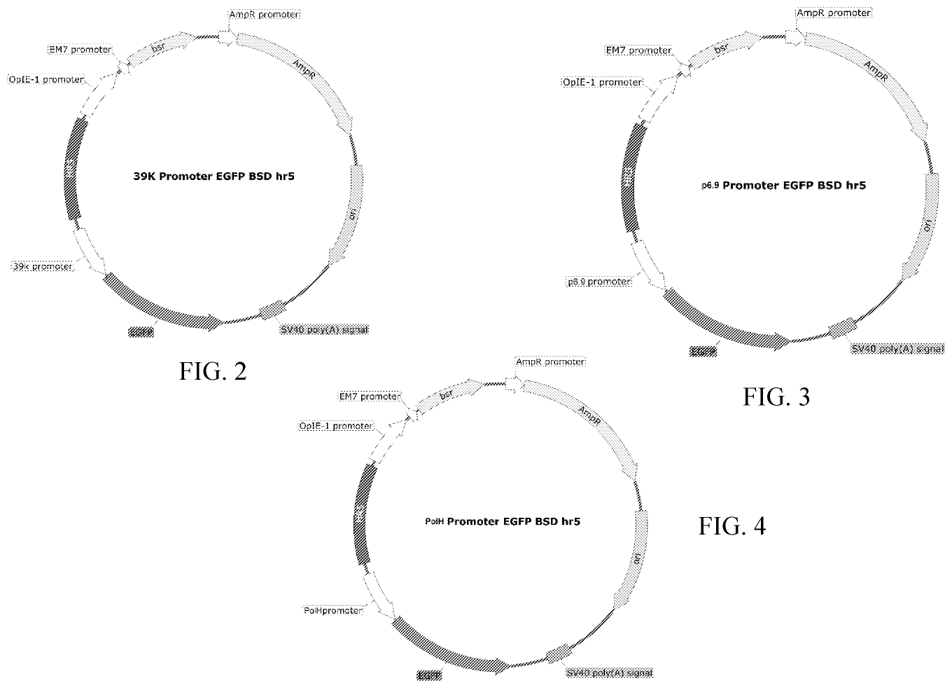

Plasmid Construction: Gibson Assembly was used to link fragments including an E. coli selection cassette, and insect cell selection cassette from pIB CMV GFP, the homologous region 5 sequence from AcMNPV E2 genome KM667940, and 39k (SEQ ID NO: 1), p6.9 (SEQ ID NO: 2), and Polh (SEQ ID NO: 3) promoters. Initially, three plasmids were constructed, each using a different promoter, either 39k (FIG. 2), p6.9 (FIG. 3), or Polh (FIG. 4). Each of the plasmids contained a reporter cassette, an Escherichia coli (E. coli) selection cassette, and an insect cell selection cassette. The reporter cassette contained either a 39k promoter, p6.9 promoter, or Polh promoter operably linked to a nucleotide sequence encoding a reporter protein (e.g., green fluorescent protein (GFP)). The E. coli selection cassette was an Ampicillin resistance cassette including an Ampicillin resistance promoter operable linked to an ampicillin resistance gene. The insect cell selection cassette contained a Blasticidin resistance gene operably linked an EM7 promoter. Later, a second set of three plasmids was constructed where the GFP gene was replaced with one optimized for expression in insect cells. Plasmid mini preparations were verified for accurate assembly, and E. coli clones carrying the correct constructs were scaled up and the plasmids were extracted and purified using maxi preparations. Additionally, constructs containing the optimized GFP gene were then linearized with ScaI before transfection.

Transfection: Transfections were performed in a 6-well plate. 2 mL/well of Sf9 cells at 0.5E6 cells/mL were plated in SF900 III to get 1e6 cells/well. 56 μL of Cellfectin II reagent was diluted in 700 μL of PBS and 3 μg of each plasmid were diluted into 100 μL of PBS. 100 μl of the diluted Cellfectin was added to each of the diluted plasmid solutions which was then incubated for 30 minutes at RT. Sf900III (0.8 ml) was then added to the plasmid solutions and the media in the plates was removed and replaced with the plasmid solutions. The plates were then incubated at 28° C. for 4-5 hours before removing the transfection mix from the wells and replacing it with 3 ml of Sf900III media with either 25 or 50 μg/ml of blasticidin, depending on the well. They were then incubated for another 72-96 hours at 28° C.

Selection: Selection was performed in media with either 25 or 50 μg/ml of blasticidin during transfection. After the cells were transferred to shake flasks, the concentration of blasticidin was left at 25 μg/ml for all cultures in order to maintain selective pressure.

Initial clone identification and screening: Splits from the shake flasks were infected with recombinant baculovirus (rBV) at a high MOI and then analyzed by flow cytometry for GFP expression at various times between 15-96 hours. Cells from the 39k-unoptGFP culture were diluted and seeded onto 96-wells plates for sub cloning. Cells were diluted to 5 cells/mL in the conditioned medium+25 μg/mL blasticidin and seeded at 200 μL/well (1 cell/well) in 20 plates. Another 10 plates were seeded in the same way, but with the addition of 1e4 feeder cells/well (untransfected Sf9 cells).

Cell Banking: Cells were seeded at 0.5E6 cells/mL on day 0 and banked on day 2 after seeding. Five vials were prepared at 30E6 cells/vial. Cells were removed from their shake flask and centrifuged at 300 g for 10 minutes at 4 C. Freezing media was prepared at a concentration of 50% fresh media and 50% spent media with 7.5% DMSO. The freezing media was filtered and 5 mL was used to re-suspend the cells. 1 mL of the cells was added to each vial and they were placed in a cryofreeze container for 24 hours at −80° C. before being transferred to the Cryotank.

Subcloning of 39k-GFP Stable Pool: Conditioned medium was harvested from naive Sf9 cells (4e6 cells/ml, day 3 culture) and sterile-filtered. Sf9 cells transfected with the 39k-GFP plasmid were diluted to 5 cells/mL in the conditioned medium+25 μg/mL blasticidin and seeded at 200 μL/well (1 cell/well) in 20 plates. Single cell clones were sorted into 96 well plates and expanded. Samples of the clones were extracted and infected with rBV at different MOI: GFP expression was measured via flow cytometry.

### Analysis of Indicator Cell Lines

The indicator cells were seeded on 96-well deep-well plates and subsequently infected with serial dilutions of baculovirus and allowed to incubate at 28° C. for 18 hours on a shaker. The indicator cells were transfected with plasmids having an GFP expression cassette containing either the 39k promoter nucleotide sequence, the p6.9 promoter nucleotide sequence, or the polyhedrin promoter nucleotide sequence.

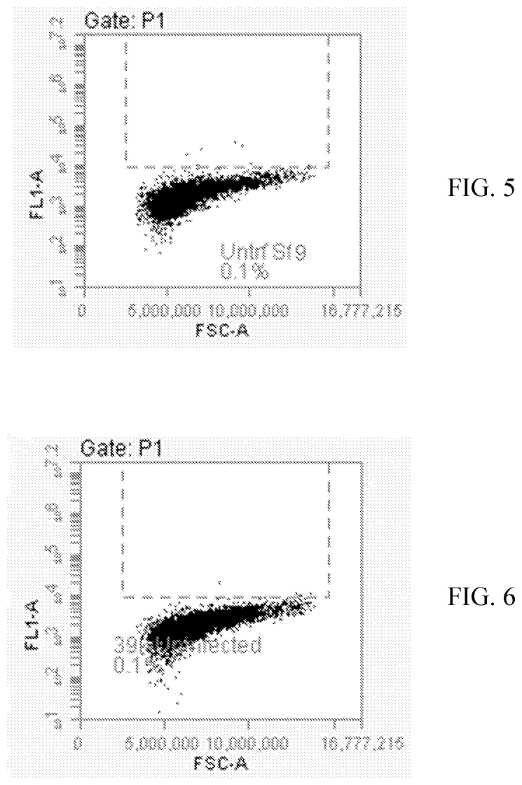

After infection, the cells were analyzed by flow cytometry for GFP expression. FIG. 5 is a graph from a flow cytometry analysis of naive Sf9 cells. FIG. 6 is a graph from a flow cytometry analysis of Sf9 cells transfected with the GFP expression cassette containing the 39k promoter nucleotide sequence. These Sf9 cells were not infected with rBV. For both figures, the dotted line shows green fluorescence and neither the naive Sf9 cells or uninfected Sf9 cells with the 39k plasmid were fluorescing. Specifically, ˜0.1% of the cells exhibited green fluorescence.

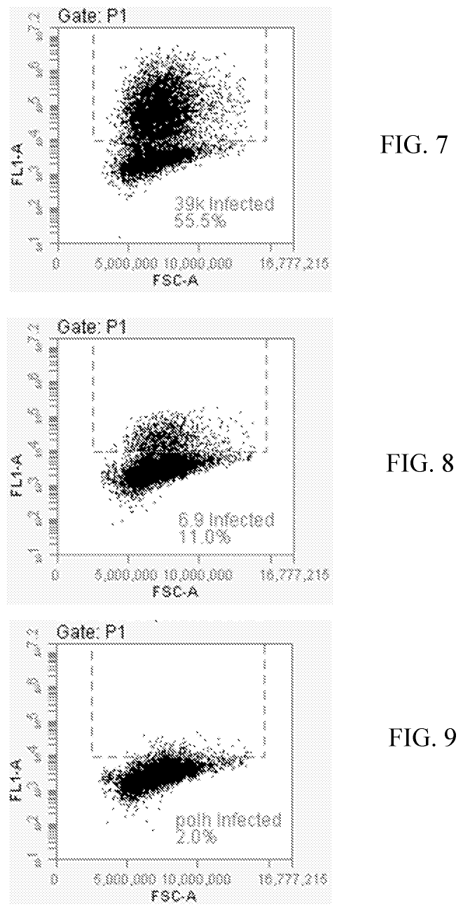

FIG. 7 is a graph from a flow cytometry analysis of Sf9 cells transfected with the GFP expression cassette containing the 39k promoter nucleotide sequence. FIG. 8 is a graph from a flow cytometry analysis of Sf9 cells transfected with the GFP expression cassette containing the p6.9 promoter nucleotide sequence. FIG. 9 is a graph from a flow cytometry analysis of Sf9 cells transfected with the GFP expression cassette containing the polyhedrin promoter nucleotide sequence. These Sf9 cells were infected with rBV. For these figures, the dotted line shows green fluorescence and the different cells exhibited fluorescence. For FIG. 7, 55.5% of the 39k promoter cells exhibited green fluorescence. For FIG. 8, 11% of the p6.9 promoter cells exhibited green fluorescence. For FIG. 9, 2% of the Polh promoter cells exhibited green fluorescence.

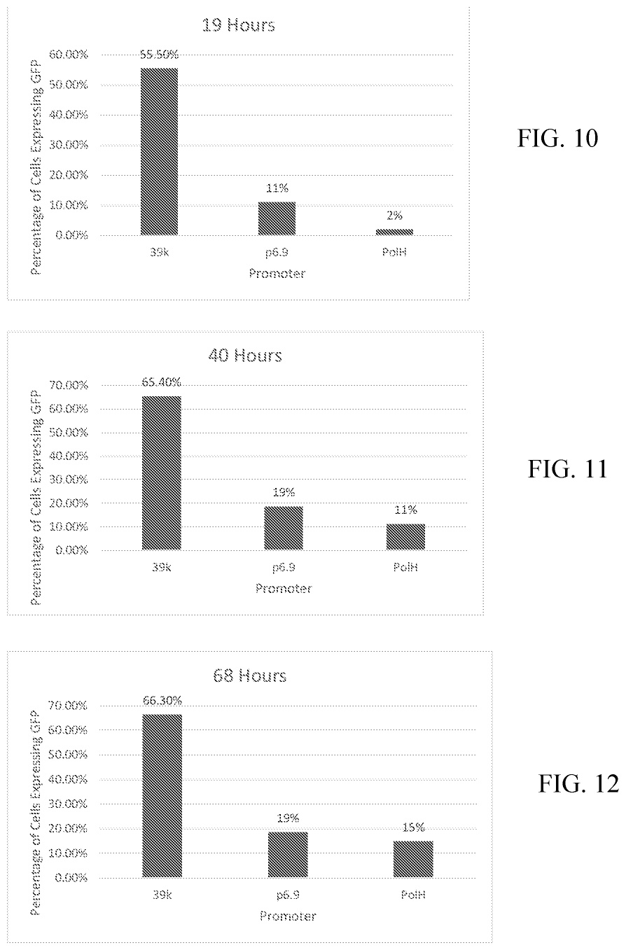

GFP expression under each promoter nucleotide sequence was measured over time. At 19 hours post rBV infection as shown in FIG. 10, 55.5% of the 39k promoter cells exhibited green fluorescence, 11% of the p6.9 promoter cells exhibited green fluorescence, and 2% of the Polh promoter cells exhibited green fluorescence. At 40 hours post rBV infection as shown in FIG. 11, 65.4% of the 39k promoter cells exhibited green fluorescence, 19% of the p6.9 promoter cells exhibited green fluorescence, and 11% of the Polh promoter cells exhibited green fluorescence. At 68 hours post rBV infection as shown in FIG. 12, 66.3% of the 39k promoter cells exhibited green fluorescence, 19% of the p6.9 promoter cells exhibited green fluorescence, and 15% of the Polh promoter cells exhibited green fluorescence. As shown in FIGS. 10, 11, and 12, the p6.9 and polyhedrin promoter sequences both show a great increase in GFP expression, but the expression is later than the 39K promoter sequences. Further, 39K promoter sequences displayed the highest GFP expression.

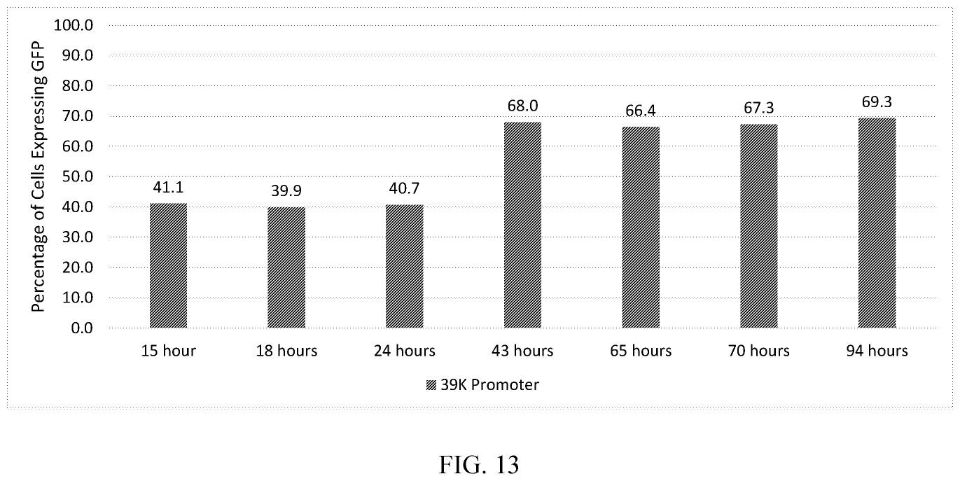

FIG. 13 shows data from an analysis of the 39k promoter, where insect cells containing the reporter cassette with the 39k promoter were analyzed for GFP expression via flow cytometry after the insect cells were infected with rBV and incubated for a predetermined time period. The percentage of 39k promoter cells expressing eGFP was 41.1% (15 hours), 39.9% (18 hours), 40.7% (24 hours), 68.0% (43 hours), 66.4% (65 hours), 67.3% (70 hours), and 69.3% (94 hours). Also as shown in FIG. 13, GFP expression under the 39k promoter is expressed as early as 15 hours after infection. It was noted that the GFP expression was maintained, which could be due to secondary rBV infections after 24 hours. To this extent, the assay may be performed before secondary rBV infections (e.g., 24 hours).

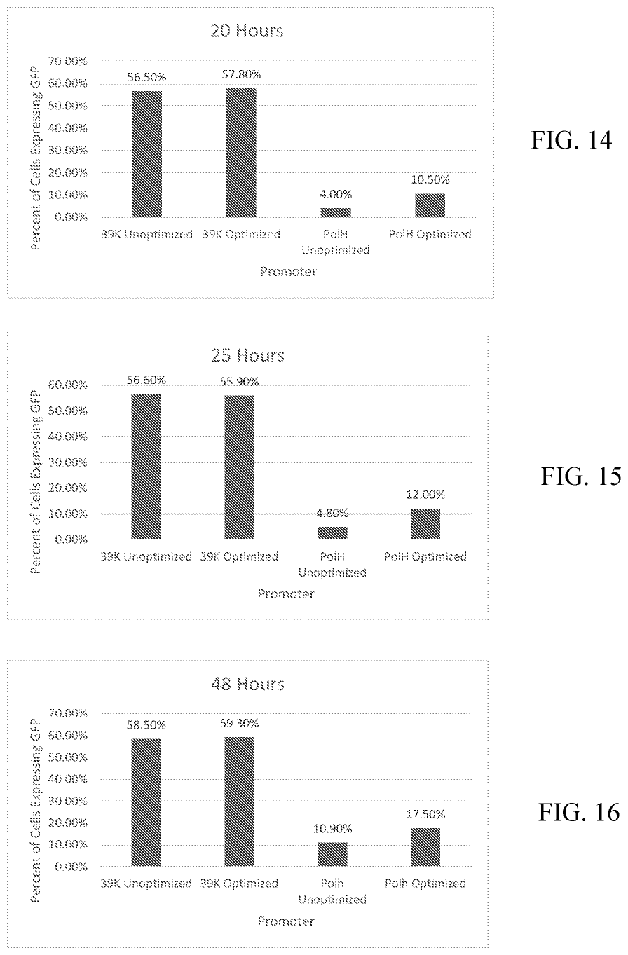

The cells were also transfected with plasmids containing nucleotide sequences encoding GFP, where the nucleotide sequences were codon optimized for GFP expression in insect cells. These nucleotide sequences were operably linked to 39K and Polh promoters. As shown in FIGS. 14, 15, and 16, the use of the codon optimized GFP nucleotide sequences increased GFP expression for the 39K and Polh promoter. At 20 hours as show in FIG. 14, use of the codon optimized eGFP sequence for 39k promoter cells increased the percentage of cells expressing eGFP from 56.5% to 57.8% and use of the codon optimized eGFP sequence for PolH promoter cells increased the percentage of cells expressing eGFP from 4.0% to 10.5%. At 25 hours as show in FIG. 15, use of the codon optimized eGFP sequence for 39k promoter cells resulted in essentially no difference eGFP expression (56.6% and 55.9%) and use of the codon optimized eGFP sequence for Polh promoter cells increased the percentage of cells expressing eGFP from 4.8% to 12.0%. At 48 hours as show in FIG. 16, use of the codon optimized eGFP sequence for 39k promoter cells increased the percentage of cells expressing eGFP from 58.5% to 59.3% and use of the codon optimized eGFP sequence for Polh promoter cells increased the percentage of cells expressing eGFP from 10.9% to 17.5%. It was still noted that GFP expression was greater for the 39K promoter.

The following is an example of rBV titering using the indicator cell line. The P0 baculovirus infectious titer is measured using a flow cytometry-based baculovirus infectious titer assay (FC-BITA) to calculate the volume of rBVs required to infect the Sf9 cells to start the production. This assay uses an Sf9 cell line that expresses GFP (Green Fluorescent Protein) upon infection with rBV. GFP levels are detected by flow cytometry and a Poisson distribution is used to convert the percentage of fluorescent cells to an rBV concentration. A positive control is run in every assay to confirm assay performance.

### Example 3

### Influence of Baculovirus MOI on AAV Productivity and Encapsidated Baculovirus-Derived DNA Profile

Insect cell-based production of recombinant adeno associated virus (rAAV) is typically achieved by infecting Sf9 cells with rBV encoding AAV Rep genes, AAV Cap genes, and a transgene of interest. The effect of rBV MOI on Sf9-produced, AAV vector yield and packaging of DNA impurities was evaluated. A full-factorial, 3-level experiment was performed in bioreactors, followed by small scale studies, to investigate the independent effects at low MOIs. A 10-fold MOI range (0.003-0.03) was investigated for all rBVs. Statistical analysis demonstrated that AAV5 productivity was positively influenced by Rep and Cap initial gene levels but negatively influenced by GOI initial levels. Similar trends were observed for total capsid production, which translated to comparable capsid-to-vector genome ratios (cp:vg) among conditions. Packaging of BV DNA impurities in AAV5 was calculated by tracking the copy number of BV genetic markers located close (Alpha and Beta) and far (Gamma and Delta) from the AAV ITRs. Results suggest that the MOI effect was dependent on the distance from the ITRs. Increasing MOIs of all rBV exerted a mild negative effect on DNA accumulation from loci close to the ITRs (Alpha:vg and Beta:vg ratios). On the other hand, DNA accumulation from loci far from the ITRs (Gamma:vg and Delta:vg ratios) was positively impacted by Rep initial gene levels and negatively impacted by GOI initial levels. The identified trends highlight the major influence the BV MOI has on vector productivity and product quality. Overall, our data suggest that higher Rep and Cap initial levels might lead to higher productivity but at the expense of an additional increase in co-packaged, rBV DNA impurities. This negative effect could be mitigated by infecting with all rBVs at a similar MOI: We speculate that packaging of rBV DNA impurities is Rep-dependent and that the levels of encapsidated rBV DNA impurities depend on Rep concentration.

### Introduction

rAAV represents one of the most promising therapeutic modalities intended to cure or mitigate the effects of a variety of monogenetic disorders. Extensive scientific evidence focusing on the understanding of the biology of AAV, as well as clinical evaluations of the safety and efficacy of rAAV, supports current efforts to make gene therapies available for patients' use [Aguti S et al. Expert Opin Biol Ther 2018; 18:681-93; Ramlogan-Steel C A et. Clin Experiment Ophthalmol 2019; 47:521-36; Li C and Samulski R J. Nat Rev Genet 2020; 21:255-72]. rAAVs have been traditionally produced in anchorage-dependent mammalian cell lines such as HEK293 by plasmid transfection. The need to improve specific productivity, process robustness and scalability led to the development of alternative cell culture processes using a variety of hosts. The insect cell/rBV system is recognized by many as one of the most scalable and productive for rAAV manufacturing. Seminal papers from Robert Kotin and Masashi Urabe established the foundations of the insect cell/BV system as an efficient means for viral vector production [Urabe M et al. Hum Gene Ther 2002; 13:1935-43; Urabe M et al. J Virol 2006; 80:1874-85]. Their approaches comprised the arrangement of AAV genes controlled by insect-specific promoters and distributed by their cis or trans-regulatory activities among two or three baculoviruses. Over time, several groups identified ways to improve the molecular design of recombinant BV, which translated to more robust vector production [Chen H. Mol Ther 2008; 16:924-30; Smith R H et al. Mol Ther 2009; 17:1888-96; Mietzsch M et al. Hum Gene Ther Methods 2017; 28:15-22].

Like for most biologics' production platforms, exhaustive process characterization is key to identify parameters that influence process performance and product quality. MOI is defined as the number of infectious rBVs divided by the total number of cells, is well known to play a significant role during recombinant protein expression. Several studies have described how varying MOI concentrations influence rBV replication dynamics, host-rBV metabolic interactions, and overall protein expression [Radford K M et al. Cytotechnology 1997; 24:73-81; Pastor A R et al. Vaccine 2019; 37:6962-9; Virag T et al. Hum Gene Ther 2009; 20:807-17]. In the context of rAAV production, this information is only partially applicable, as subsequent vector-specific molecular events (e.g., capsid assembly, rAAV DNA replication and packaging) must take place after protein expression to generate infectious vector particles [Aponte-Ubillus J J et al. Appl Microbiol Biotechnol 2018; 102:1045-54]. There are a handful of studies evaluating the effect of MOI on recombinant AAV production. Meghrous and Aucoin evaluated the effect of total MOI and MOI ratios using 3 rBVs to provide AAV genes. The initial assessment highlighted the benefits of using high MOI strategies (MOI>3), and the importance of a balanced BV MOI ratio for high productivity [Meghrous J et al. Biotechnol Prog 2005; 21:154-60]. A later report augmented the study on high MOI strategies and confirmed the positive effect of Rep BV and Cap BV MOI on infectious vector yield [Aucoin M G et al. Biotechnol Bioeng 2006; 95:1081-92]. There is a lack of studies characterizing asynchronous, low MOI BV infections in rAAV production processes. In low MOI infections, a small fraction of cells is infected after virus addition. Secondary infection rounds are a result of viral replication and lead to infection of the totality of the cell population. Mena et al. [Mena J A et al. J Gene Med 2010; 12:157-67] described comparable AAV infectious yield when using either a low MOI (0.3) or high MOI (9) in a 3-rBV process. Additional optimization of seeding cell density and feeding strategy led to further yield increase. Less understood is the effect of BV MOI on rAAV product quality. Vector quality is as important as vector productivity, because it assures robust expression and activity of the AAV-derived transgene. Packaging of DNA impurities is a phenomenon that has been documented during rAAV production, where sequences derived from helper plasmids or BV DNA get erroneously encapsidated [Chadeuf G et al. Molecular Therapy 2005; 12:744-53; Wright J F. Biomedicines 2014; 2:80-97]. Studies have reported that plasmid backbone and rBV backbone sequences could be present in rAAV vector stocks at percentages as high as 6% and 3%, respectively [Lecomte E al. Molecular Therapy—Nucleic Acids 2015; 4:e260; Penaud-Budloo M et al. Hum Gene Ther Methods 2017; 28:148-62]. In the context of insect cell systems, it is believed that not only rBV molecular design, but also upstream process parameters could play a role in packaging of DNA impurities. More studies need to be performed to obtain clues on the contribution of biological inputs and cell culture parameters to the formation of this product-related impurity.

At large scale, low rBV MOI strategies could simplify BV expansion operations, reduce operational costs, and improve BV genetic stability. Therefore, the understanding of the implications of low MOI strategies on rAAV generation gains importance. In the present study, we investigated how different low BV MOIs and gene initial ratios influence productivity and rAAV quality, by monitoring specific outputs such as per-cell productivity, capsid-to-vector genome ratio, and packaging of rBV-derived DNA impurities. A follow-up evaluation was executed, building a hypothesis that could explain the identified trends.

### Material and Methods

### Cell Line and Culture Maintenance

A subclone from Spodoptera frugiperda cell line Sf9 was used for the present study. Cells were passaged twice a week in shake flasks (Corning, NY) containing a proprietary serum-free medium, targeting an initial cell density of 5×10⁵ cells/mL. Shake flasks were incubated at 28° C.

### Recombinant BV Generation

Recombinant bacmids and rBVs were designed and produced using the bac-to-bac expression system (Thermo Fisher Scientific, CA). Bac-GOI-A (transgene A) and Bac-GFP-GOI-B (Transgene B) contained ITR-flanked transgenes of 4.6 and 4.8 kb of length, respectively. Bac-GFP-GOI-B contained the GFP gene controlled by GP64 promoter, in addition to transgene B. Baculoviruses were designed to express Rep (e.g., Rep78 and Rep52) and Cap genes via different baculovirus promoters. An additional construct contained a dTomato fluorescent protein expression cassette regulated by another baculovirus promoter. Quantitation of infectious BV was determined by flow cytometry, using a Sf9-derived indicator cell line that expresses GFP under the control of the 39k promoter. This titration method has been evaluated against other well-established protocols to assure the accuracy of the multiplicity of infection values (MOIs) used during the subsequent experiments (data not shown).

### Design of Experiments

A bioreactor study was performed to assess the effect of rBV MOI on AAV5 productivity and BV-derived encapsidated DNA impurities. A full factorial experimental matrix was designed with JMP 14 (SAS). The MOI evaluation range was defined as one log, to prevent process variability due to significant differences in cell growth or nutrient consumption. The amount of BV volume added per vessel represented a fraction lower than 0.1% of the working volume (3L) in all cases. A Dasgip controller (Eppendorf, CT) was used to operate the bioreactors. All seeding cell density, infection time, harvest time, and physicochemical parameters (pH, DO, Temperature) were consistent among conditions. Supernatant underwent chemical treatment to promote additional rAAV particle release and clear process-related impurities. Centrifugation at 4000×g for 15 minutes and depth filtration was performed to further clarify the harvest material.

A follow-up study in shake flasks was carried out to generalize trends observed during the bioreactor study. Two different GOI BV were tested. 125 mL shake flasks were used to test four representative conditions identified in the previous study. An inoculation, infection and harvest schedule identical to the bioreactor study was followed, obtaining clarified harvest as final upstream material.

### AAV Affinity Purification

Aliquots of each clarified harvest material were incubated with a slurry of AVB Sepharose resin (Thermo Fisher Scientific, CA) for 2 hours at room temperature and constant agitation. Each AVB resin/harvest mixture was then centrifuged, and the pelleted resins were transferred to Acroprep filter plates (Pall Corporation, NY), where they were processed in parallel. Resins were washed three times with phosphate buffer solution and incubated with a low-pH buffer for 3.5 minutes to elute rAAV capsids. Liquid contents were removed from the filter plate to a 96 deep-well plate with the use of a multi-plate vacuum manifold (Pall Corporation, NY). Collected eluates were pH-adjusted to 7.0-7.2 before storage.

DNA Quantification by Digital Droplet PCR (ddPCR)

The presence of capsid-protected transgene and BV-derived DNA impurities was tracked by ddPCR, following the protocol described in Barajas et al. [Barajas D et al. PLoS One 2017; 12]. Serial dilutions of AVB eluates were performed to cover the wide concentration range of the target sequences tested. Dilutions that resulted in less than—5000 copies per microliter of reaction were used for quantification. Appropriate non-template controls showed copy number lower than 1 at all times. An automated droplet generator and reader (Bio-Rad Laboratories, CA) were used. Detection of positive droplets and copy number determination was performed by Quantasoft software (Bio-Rad Laboratories, CA). To determine VP3/18s ratio from cells, 2 mL of cell culture was spun down at 500×g for 2 minutes, and the cell pellet was recovered and frozen at −80° C. Frozen pellets were later resuspended in TE buffer+0.5% SDS, and incubated at room temperature for 1 hour. This suspension was used as starting material for ddPCR quantification.

### Capsid Quantification

An Octet system (Molecular Devices, CA)-based high-throughput method was used to quantify total capsids based on a standard curve built from antibody-capsid binding kinetics information using AAV5 standard material at different concentrations. Dilutions were performed to meet the assay's dynamic range. Each sample was analyzed in duplicates, along three dilutions. A positive control was included to track the assay's precision.

### Flow Cytometry Analysis

Attune NxT (Thermo Fisher Scientific, CA) was used to monitor the percentage of GFP-expressing and dTomato-expressing cells over culture time. Channels YL1 and BL1 were used to track the different signals. One million cells per condition were collected per sample run on the analyzer. GFP-positive, dTomato-positive, and negative (uninfected) controls were included during the analysis. Samples were taken at different time points post BV infection. At least 20,000 events per sample were analyzed to calculate the infection percentages.

### Results

A preliminary study performed in bioreactors producing AAV-transgene A. It was decided to cover a 10-fold MOI range to minimize the impact on cell culture growth performance caused by varying viral load. The viability and growth rate trends support the claim that potential variability in cell growth and death trends among tested conditions is insignificant and should not impact the conclusions made around the effect of BV MOI on productivity and product quality.

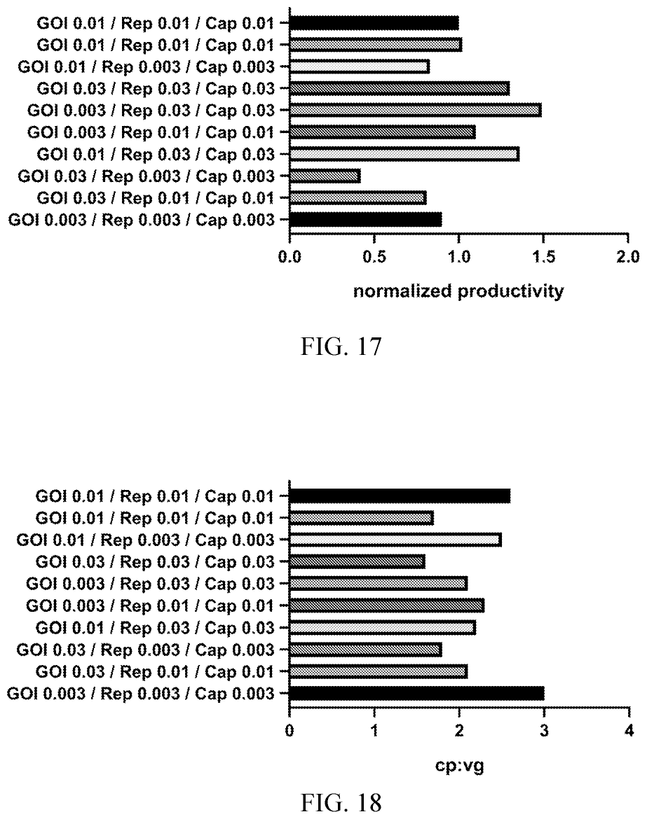

Clarified harvest and affinity-purified material were assayed for rAAV-transgene A vg titer and capsid-to-vector genome (cp:vg) ratio. Productivity was normalized and is shown in FIG. 17. The highest productivity value was obtained when AAV genes were provided at rBV MOIs of: GOI 0.003/Rep 0.03/Cap 0.03, whereas the lowest value was obtained when they were provided at the initial gene levels GOI 0.03/Rep 0.003/Cap 0.003. A statistical model was developed to describe the effect of MOI of rBVs providing/encoding GOI, Rep, and Cap and their interaction on per-cell productivity. Cp:vg ratios range from 1.5-3 among tested conditions (FIG. 18). It was initially hypothesized that conditions with higher Rep and Cap BV MOI might show higher ratios due to increased likelihood of empty capsid production; however, that phenomenon was not evidenced in this experiment. It is plausible that an evaluation range larger than 10-fold could detect significant differences in encapsidation efficiency.

The influence of rBV MOI on the quality of rAAV material was characterized by quantifying four specific nuclease-resistant, rBV-derived genetic markers present in purified product. Markers Alpha and Beta are located within a 10 kilobase (kb) region adjacent to the AAV ITRs but external to the rAAV vector genome nucleotide sequence in the baculovirus genome, whereas markers Gamma and Delta are distant from the ITRs, covering approximately 135 kb of BV DNA genome. Table 1 displays results determined as marker:vg ratio and as a percentage of rBV-derived cDNA impurities. Table 1 shows the effect of rBV MOI on encapsidation of rBV-derived DNA impurities. Experimental conditions are presented based on individual rBV MOI and rBV MOI ratio. Ratios were averaged (Alpha-Beta, Gamma-Delta) and normalized to condition #10. In addition, the percentage of rBV-derived DNA impurities present in purified vectors was inferred, using the methodology from Penaud-Budloo [Grosse S et al. J Virol 2017; 91] as reference. The percentage of DNA contaminants was calculated from the copy number of the rAAV transgene, averaged Alpha-Beta, and averaged Gamma-Delta; and normalized to each reference size (AAV transgene=4.8 kb; Alpha-Beta near-ITR region=10 kb; Gamma-Delta backbone region=135 kb). An averaged AlphaBeta:vg or Gamma-Delta:vg ratio was used to provide a more representative estimate of the packaging of BV-derived DNA impurities frequency from each region. We estimated that increasing Rep and Cap levels contribute to lower concentration of markers that surround the ITRs within a 10 kb section. At low GOI levels (0.003), a variation of Rep and Cap BV MOI levels from low (0.003) to high (0.03) reduces the normalized Alpha-Beta DNA ratio from 1.55 to 0.75 (52% decrease). Moreover, the concentration patterns for Gamma-Delta markers were negatively affected by GOI BV MOI: The normalized Gamma-Delta:vg concentration within the evaluated conditions ranges from 0.98 to 16.09, suggesting that BV MOI has a stronger influence on genetic sequences that are far from the ITR, which are less likely to be part of reverse-packaging events. The estimation of the percentages of BV-derived DNA impurities in rAAV particles showed total (Alpha-Beta+Gamma-Delta) values in a range from 0.22-0.60%, which aligned with previous reports [Penaud-Budloo M et al. Hum Gene Ther Methods 2017; 28:148-62]. Overall, it is suggested that higher Rep and Cap initial levels might lead to higher productivity, but at the expense of an additional increase in BV-derived DNA impurities. This negative effect could be mitigated by keeping rBV ratios closer to 1.

TABLE 1

| | | | | Packaging of BV DNA impurities | | | | |
|---|---|---|---|---|---|---|---|---|
| | | | | Normalized | Normalized | % rBV | % rBV | % rBV |
| BV Ratios | | | | Alpha-Beta:vg | Gamma-Delta:vg | backbone | backbone | backbone |
| Condition | GOI | Rep | Cap | ratio | ratio | Alpha-Beta | Gamma-Delta | total |
| 1 | 0.003 | 0.03 | 0.03 | 0.75 | 16.09 | 0.18 | 0.42 | 0.6 |
| 2 | 0.003 | 0.01 | 0.01 | 1.17 | 9.55 | 0.28 | 0.25 | 0.53 |
| 3 | 0.01 | 0.03 | 0.03 | 0.99 | 7.09 | 0.24 | 0.19 | 0.43 |
| 4 | 0.003 | 0.003 | 0.03 | 1.55 | 4.68 | 0.38 | 0.12 | 0.5 |
| 5 | 0.03 | 0.03 | 0.03 | 0.76 | 1.48 | 0.18 | 0.04 | 0.22 |
| 6 | 0.01 | 0.01 | 0.01 | 0.96 | 2.28 | 0.23 | 0.06 | 0.29 |
| 7 | 0.01 | 0.01 | 0.01 | 1.27 | 3.21 | 0.31 | 0.08 | 0.39 |
| 8 | 0.03 | 0.01 | 0.01 | 0.99 | 0.98 | 0.24 | 0.03 | 0.27 |
| 9 | 0.01 | 0.003 | 0.003 | 1.28 | 2 | 0.31 | 0.05 | 0.36 |
| 10 | 0.03 | 0.003 | 0.003 | 1 | 1 | 0.24 | 0.03 | 0.27 |

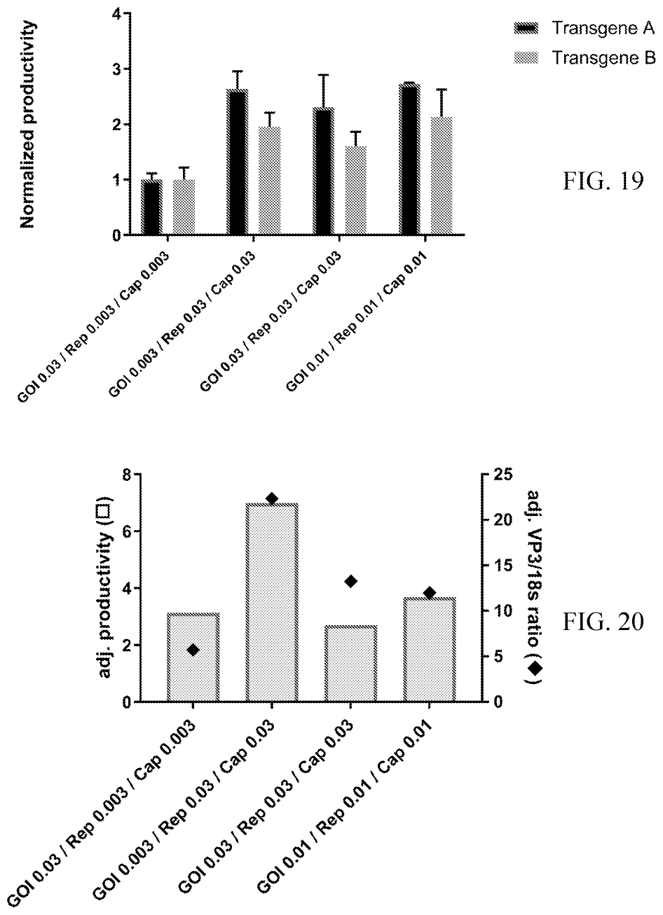

A follow-up study in shake flasks was performed to increase the understanding of the productivity and trends in BV-derived DNA impurities. Different BV MOI conditions were replicated using different BV sets: rBVs providing/encoding GOI-A, Rep, and Cap (same as above) and fluorescently labeled dTomato-rBVs providing/encoding Rep, Cap, and GFP-GOI-B, to confirm the previous productivity trends. All conditions infected with the fluorescent protein-producing BV set were monitored using flow cytometry and ddPCR to identify potential correlation among rBVs encoding Rep or Cap infection levels, Cap expression and productivity. Flow cytometry analysis highlighted the percentage of co-infected cells at 90 hours post-infection (hpi) during production of AAV-GFP-GOI-B. A significant imbalance in the BV MOI ratio can lead to low coinfection percentages (32.2% and 28.9% for GOI 0.03/Rep 0.003/Cap 0.003 and GOI 0.003/Rep 0.03/Cap 0.03 conditions, respectively), whereas conditions with a BV ratio of 1:1:1 showed coinfection percentages between 68.8-70.6%. Compared to insect cell/BV processes aimed at protein production, successful generation of rAAV particles requires cells to be co-infected with all BVs. Therefore, the BV coinfection rate can theoretically influence per cell productivity. FIG. 19 shows comparable productivity among conditions with a GOI/Rep/Cap BV MOI ratio of 1:1:1 or lower (e.g., 1:<1:<1), irrespective of the transgene identity. Because previous results suggested the positive influence of MOI of rBV encoding Rep or Cap on vector yield, VP3 gene copy number in cell pellets post-infection was measured. In this instance VP3 serves as a proxy for cellular Rep or Cap copy number. Host cell 18s ribosomal RNA gene marker was also tracked to account for different cell densities. FIG. 20 compares AAV-GOI-B productivity and VP3/18s ratios against various BV MOI combinations. The productivity and VP3/18s ratios were adjusted based on the assumption that only co-infected cells produced “full” AAV particles, and that only cells infected with rBV encoding Rep or Cap contain detectable levels of AAV VP3 DNA. These results showed a positive correlation between adjusted VP3/18s DNA ratio and adjusted per-cell productivity. Altogether, results from FIGS. 19 and 20 confirm the strong influence MOI of rBV encoding Rep or Cap exerts on productivity. Although conditions operating at GOI/Rep/Cap rBV MOI ratio of lower than 1:1:1 (e.g., 1:<1:<1) appear to co-infect a lower number of cells, this subpopulation contains a higher Rep or Cap copy number. This effect appears to improve vector productivity in that specific subpopulation, bringing the bulk cell productivity to levels similar to conditions with BV MOI ratios equaling 1:1:1.

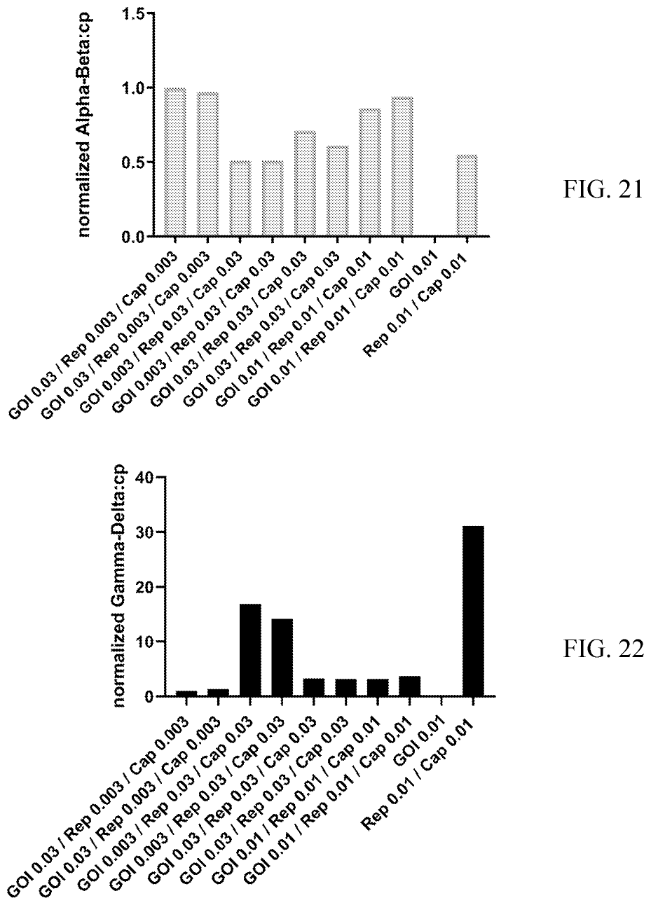

Finally, the effect of MOI of rBV encoding Rep or Cap on encapsidation of BV-derived DNA impurities was confirmed. Vector particles made from the distinct dTomato-rBVs providing/encoding Rep, Cap, and GFP-GOI-B MOI conditions were purified and the level of BV-derived DNA impurities per capsid (res DNA:cp ratio) was determined. FIGS. 21 and 22 display normalized, BV-derived DNA:cp ratios for averaged Alpha-Beta and Gamma-Delta markers, respectively. Similar to the bioreactor study, increase in MOI of rBV encoding Rep or Cap has a negative impact on Alpha-Beta:cp ratio, leading to a 50% reduction when switching from a GOI/Rep/Cap BV MOI ratio of 10 (e.g., 10:1:1) to 0.1 (e.g., 0.1:1:1). Concentrations of BV-derived DNA impurities in capsids produced with only rBV encoding Rep or Cap aligned well with this accumulation trend. The switch from a GOI/Rep/Cap BV MOI of 10 to 0.1 led to approximately 15-fold increase in Gamma-Delta DNA:cp ratio, and up to a 30-fold increase seen in rAAV particles produced with rBV encoding Rep or Cap only. Moreover, conditions infected with a GOI/Rep/Cap BV MOI ratio of 1, regardless of the precise MOI, showed comparable results. In addition, the % rBV encoding Rep or Cap-only infected cells at 90 hpi negatively correlates with Alpha-Beta DNA:cp ratio, and positively correlates with Gamma-Delta DNA:cp ratio. Overall, these results suggest BV MOI ratio imbalances leaning towards higher MOI of rBV encoding Rep or Cap lead to a shift in cell subpopulations where an increasing percentage of cells might be producing transgene-free capsids, and the disproportional accumulation of BV-derived DNA in transgene-free capsids might translate into a global increase of BV-derived DNA impurities in rAAV capsids. These results also confirmed the contrasting trends in encapsidation of BV-derived DNA impurities depending on the location of the markers in the BV genome.

The concept of low rBV MOI infection strategy brings important advantages for viral stock preparation. The 100 to 1000-fold reduction in BV stock represents a significant operational relief in baculovirus generation, which becomes of ultimate importance when operating at large scales [Virag T et al. Hum Gene Ther 2009; 20:807-17]. It also has a positive impact on BV genetic stability, as lower BV inoculum minimizes the generation of defective-interfering particles as a result of the “passage effect” during inoculum expansion [Krell P J. Cytotechnology 1996; 20:125-37]. In the context of rAAV manufacturing, the utility of low BV MOI strategies during insect cell/BV operations justifies a thorough investigation on how varying MOIs could impact the yield and quality of the vector particles.

As total BV MOI decreases, the percentage of cells co-infected during the initial viral infection round decreases. The subsequent asynchronous infection process is influenced by other inputs such as cell line behavior, number of BVs used, and time of infection [Mena J A et al. BMC Biotechnol 2007; 7:39; Lee D F et al. J Virol 2000; 74:11873-80; Sokolenko S et al. Biotechnol Adv 2012; 30:766-81]. Physicochemical parameters such as culture temperature also exert an effect on the timing of AAV protein expression and vector production, suggesting this parameter might have an impact on BV replication and cell death kinetics [Aucoin M G et al. Biotechnol Bioeng 2007; 97:1501-9]. The present study explored varying MOI values for rBVs containing Rep, Cap and GOI sequences, while all other process parameters remained constant. Data analysis highlights the positive effect of Rep and Cap genes on vg productivity during the infection process. This result aligns with previous experiments performed by Meghrous and Aucoin at high MOIs [Meghrous J et al. Biotechnol Prog 2005; 21:154-60; Aucoin M G et al. Biotechnol Bioeng 2006; 95:1081-92]. Successful infection with Rep and Cap BV promotes strong expression of Rep proteins necessary for rAAV DNA replication, genome resolution and packaging into pre-formed capsids [Samulski R J and Muzyczka N. Annual Review of Virology 2014; 1:427-51]. It also boosts expression of AAV VP proteins and the assembly-activating protein (AAP), the latter being important for chaperoning protein transport for proper capsid assembly [Grosse S et al. J Virol 2017; 91]. A review of the relevant literature showed that operating at a BV MOI ratio of 1 is preferred, as it leads to consistent process performance. The results obtained support that rule of thumb; however, they also contribute to the notion that there is flexibility over that ratio. Aucoin [Aucoin M G et al. Biotechnol Bioeng 2006; 95:1081-92] showed that reducing GOI:Rep:Cap BV ratio from 10:10:10 (total MOI of 30) to 3:10:10 (total MOI of 23) lead to comparable infectious titer results, suggesting that the initial number of transgene (GOI) copies provided in synchronous infection processes is required in lower amounts relative to the Rep and Cap copy numbers. While not being bound to this theory, the inventors hypothesize that, in conditions of low initial GOI copy numbers, Rep-driven transgene replication can supply abundant ITR-flanked DNA for subsequent packaging. In conditions with lower GOI but high Rep and Cap levels, flow cytometry data suggest that both the percentage of cells infected by the virus and superinfection levels are potentially impacted (data not shown). Interestingly, such a ratio improved the bulk cellular productivity.

Encapsidation of BV-derived DNA impurities was also assessed in the present study. In Sf9 production systems, evaluation of DNA impurities by next generation sequencing and PCR-based techniques identified BV and host cell-derived DNA sequences at total percentages ranging from 0.2-2% of the genome, BV DNA being the most abundant [Penaud-Budloo M et al. Hum Gene Ther Methods 2017; 28:148-62; Kondratov O et al. Mol Ther 2017; 25:2661-75]. Although DNA impurities are present at a small percentage, regulatory health authorities advise manufacturers to control this product-related impurity to reduce any potential genotoxicity risk [FDA Briefing Document: Vaccines and Related Biological Products Advisory Committee Meeting: Sep. 19, 2012: Cell Lines Derived from Human Tumors for Vaccine Manufacture n.d.:30]. It is believed that upstream and downstream rAAV production operations have an influence on DNA impurity levels in the drug substance. However, there is a lack of studies evaluating these hypotheses. This is believed to be the first report that systematically characterizes the effect of BV MOI on BV-derived, packaged DNA impurities in insect cell cultures. Preliminary results suggest increasing Rep and Cap BV MOI relative to the GOI MOI lowers the packaging of BV DNA from ITR-adjacent loci, while increasing encapsidation of loci distant from ITRs; and these effects are not only contrasting but different in intensity. These data describe two potential mechanisms of BV-derived DNA impurities packaging: 1) the previously reported “reverse packaging”, which is highly influenced by the presence of ITR sequences; and 2) a Rep-dependent mechanism that applies to all baculovirus genome sequences. While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

1. A method of producing recombinant adeno-associated virus (rAAV), the method comprising the steps of:
infecting cells with at least one passage 0 (P0) recombinant baculovirus (rBV), wherein the at least one P0 rBV has nucleotide sequences for generating rAAV; and
culturing the infected cells to generate rAAV;
wherein prior to the infecting step, the at least one P0 rBV is isolated from at least one cell culture comprising cells transfected with at least one of the nucleotide sequences.

2.-3. (canceled)

4. A method of producing recombinant adeno-associated virus (rAAV), the method comprising the steps of:
infecting cells with recombinant baculovirus (rBV) at a multiplicity of infection (MOI) of less than 0.01, wherein the rBV has nucleotide sequences for generating rAAV; and
culturing the infected cells to generate rAAV.

5. The method of claim 4, where in the MOI is 0.002 or less.

6. The method of claim 4, where in the MOI is less than 10 E-4.

7. The method of claim 4, where in the MOI is less than 10 E-5.

8. The method of claim 4, wherein the rBV is passage zero (P0) rBV.

9. The method of claim 4, wherein the rBV comprises a first rBV having a nucleotide sequence for an rAAV vector genome and one or more second rBV having nucleotide sequences encoding Rep and Cap proteins and the cells are infected at a ratio of the first rBV MOI: the one or more second rBV MOI ranging from 0.01 to 10.0.

10. The method of claim 4, wherein the generated rAAV has a concentration of encapsidated baculoviral nucleotide sequences that is less than 1E-9 nanograms per nanogram of encapsidated rAAV vector genome.

11. The method of claim 4, wherein the generated rAAV has a concentration of encapsidated baculoviral nucleotide sequences that encodes at least a portion of a baculoviral DNA polymerase that is less than 1E-2 copies per copy of encapsidated rAAV vector genome.

12. The method of claim 4, wherein the generated rAAV has a concentration of encapsidated cellular 18S ribosomal RNA gene nucleotide sequences that is less than 1E-3 copies per copy of encapsidated rAAV vector genome.

13.-26. (canceled)

27. The method as in claim 1, wherein the cells are insect cells.

28. The method as in claim 1, wherein the cells are insect cells derived from Spodoptera frugiperda, Aedes albopictus, Bombyxmori, Trichoplusia ni, Ascalapha odorata, Drosphila, Anophele, Culex, or Aedes.

29. The method as in claim 1, wherein the cells are Sf9 cells, High Five cells, Se301 cells, SeIZD2109 cells, SeUCR1 cells, Sf900+ cells, Sf21 cells, BTI-TN-5B1-4 cells, MG-1 cells, Tn368 cells, HzAm1 cells, BM-N cells, Ha2302 cells, Hz2E5 cells, or Ao38 cells.

30.-41. (canceled)

42. A composition comprising recombinant adeno-associated virus (rAAV), wherein the rAAV has at least one of:
a concentration of encapsidated baculoviral nucleotide sequences that is less than 1E-9 nanograms per nanogram of encapsidated rAAV vector genome.
a concentration of encapsidated baculoviral nucleotide sequences that encodes at least a portion of a baculoviral DNA polymerase that is less than 1E-2 copies per copy of encapsidated rAAV vector genome;
a concentration of encapsidated cellular 18S ribosomal RNA gene nucleotide sequences that is less than 1E-3 copies per copy of encapsidated rAAV vector genome.

43. The composition of claim 42, wherein the concentration of encapsidated baculoviral nucleotide sequences that is less than 1E-9 nanograms per nanogram of encapsidated rAAV vector genome.

44. The composition of claim 42, wherein the concentration of encapsidated baculoviral nucleotide sequences that encodes at least a portion of a baculoviral DNA polymerase that is less than 1E-2 copies per copy of encapsidated rAAV vector genome.

45. The composition of claim 42, wherein the concentration of encapsidated cellular 18S ribosomal RNA gene nucleotide sequences that is less than 1E-3 copies per copy of encapsidated rAAV vector genome.
